(19) United States
(12) Patent Application Publication
(10) Pub. No.: US 2020/0101176 A1
(43) Pub. Date: Apr. 2, 2020

(54) Small Highly Uniform Nanomedicine Compositions for Therapeutic, Imaging and Theranostic Applications

(71) Applicant: THE REGENTS OF THE UNIVERSITY OF MICHIGAN, Ann Arbor, MI (US)

(72) Inventors: Thomas Hopkins, Sylvania, OH (US); Scott D. Swanson, Ann Arbor, MI (US); Raoul Kopelman, Ann Arbor, MI (US)

(21) Appl. No.: 16/570,784

(22) Filed: Sep. 13, 2019

Related U.S. Application Data
(60) Provisional application No. 62/730,882, filed on Sep. 13, 2018.

(51) Int. Cl. A61K 49/18; A61N 5/06; A61B 5/055

(52) U.S. Cl. CPC A61K 49/1818; B82Y 5/00; A61B 5/055; A61N 5/062

(57) ABSTRACT

A targetable nanoconstruct capable of simultaneously serving as a therapeutic platform for photodynamic therapy as well as an MR molecular imaging agent, free of heavy metal atoms. F3-cys targeting agent nanoconstructs, including 8PEGA-Ce6 NCs. A label-free 8PEGA nanoconstruct that can be directly and selectively imaged by MRI, using standard spin-echo imaging sequences with large diffusion magnetic field gradients to suppress the water signal.

### CROSS-REFERENCE TO RELATED APPLICATIONS

This application claims benefit of priority to and claims under 35 U.S.C. § 119(e)(1) the benefit of the filing date of U.S. provisional application Ser. No. 62/730,882 filed Sep. 13, 2018, the entire disclosure of which is incorporated herein by reference.

### STATEMENT OF GOVERNMENT INTEREST

This invention was made with government support under CA186769 awarded by the National Institutes of Health. The government has certain rights in the invention.

### BACKGROUND

### Field of the Invention

The present inventions relate generally to nanoconstructs and uses of these constructs in dynamic therapies, imaging, diagnostics, theranostics and other applications.

The terms “nanoparticle”, “nanomaterial”, “nanoparticle”, nanoproduct”, “nanoplatform”, “nanoconstruct”, “nanocomposite”, “nano”, and similar such terms, unless specified otherwise, are to be given their broadest possible meaning, and include particles, materials and compositions having a volumetric shape that has at least one dimension from about 1 nanometer (nm) to about 100 nm. Preferably, in embodiments, these volumetric shapes have their largest cross section from about 1 nm to about 100 nm.

The terms “nanoconstructs”, “nanoplatform”, “nanocomposite”, and “nanoconstruct” and similar such terms, unless specified otherwise, are to be given their broadest possible meaning, and include a particle having a backbone material, e.g., a cage, support or matrix material, and one or more additives, e.g., agents, moieties, compositions, biologics, and molecules, that are associated with the backbone. Generally, the backbone material can be a nanoparticle. Generally, the additive is an active material having targeting, therapeutic, imaging, diagnostic, theranostic or other capabilities, and combinations and variations of these. In embodiments, the backbone material can be an active material, having targeting, therapeutic, imaging, diagnostic, theranostic or other capabilities, and combinations and variations of these. In embodiments both the additive and the backbone material are active materials. One, two, three or more different types of backbone materials, additives and combination and variations of these are contemplated.

The term “theranostic”, unless specified otherwise, is to be given its broadest possible meaning, and includes a particle, agent, construct, or material that has multiple capabilities and functions, including both imaging and therapeutic capabilities, both diagnostic and therapeutic capabilities, and combinations and variations of these and other features such as targeting.

The terms “imaging”, “imaging agent”, “imaging apparatus” and similar such terms, unless specified otherwise, should be given their broadest possible meaning, and would include apparatus, agents and materials that enhance, provide or enable the ability to detect, analyze and visualize the size, shape, position, composition, and combinations and variations of these as well as other features, of a structure, and in particular structures in animals, mammals and humans. Imaging agents would include contrast agents, dies, and similar types of materials. Examples of imaging apparatus and methodologies include: x-ray; magnetic resonance; computer axial tomography scan (CAT scan); proton emission tomography scan (PET scan); ultrasound; florescence; and, photo acoustic.

The term, “diagnostic”, unless specified otherwise, is to be given its broadest possible meaning, and would include identifying, determining, defining and combinations and variations of these, conditions, diseases and both, including conditions and diseases of animals, mammals and humans.

The term “therapeutic” and “therapy” and similar such terms, unless specified otherwise, are to be given their broadest possible meaning and would include addressing, treating, managing, mitigating, curing, preventing, and combinations and variations of these, conditions and diseases, including conditions and disease of animals, mammals and humans.

The terms “photodynamic therapy”, “PDT”, “photosensitizer”, “PS” and similar such terms, unless expressly stated otherwise, are to be given their broadest possible meaning and would include a method for ablating, e.g., killing, biological tissue by photo-oxidation utilizing photosensitizer (PS) molecules. When the photosensitizer is exposed to a specific wavelength of light, it produces a form of oxygen that kills nearby cells, e.g., reactive oxygen species (“ROS”), which includes any form of oxygen that are cyto-toxic to cells. It being understood that while light across all wavelengths, e.g., UV to visible to IR, is generally used as the activator of the PS.

The terms “activation dynamic therapy”, “dynamic therapy”, “dynamic therapy agent” and similar such terms should be given their broadest possible meaning and would include PDT and PS, as well as agents that are triggered to product active oxygen, such as a reactive oxygen species (“ROS”) or other active therapeutic materials, when exposed to other energy sources other than light, as activators can be used. These would include materials or agents that are activated by energy sources such as radio waves, other electromagnet radiation, magnetism, and sonic (e.g., Sonodynamic therapy or SDT).

As used herein, unless stated otherwise, room temperature is 25° C. And, standard ambient temperature and pressure is 25° C. and 1 atmosphere. Unless expressly stated otherwise all tests, test results, physical properties, and values that are temperature dependent, pressure dependent, or both, are provided at standard ambient temperature and pressure, this would include viscosities.

Generally, the term “about” and the symbol “˜” as used herein unless stated otherwise is meant to encompass a variance or range of ±10%, the experimental or instrument error associated with obtaining the stated value, and preferably the larger of these.

As used herein unless specified otherwise, the recitation of ranges of values herein is merely intended to serve as a shorthand method of referring individually to each separate value falling within the range. Unless otherwise indicated herein, each individual value within a range is incorporated into the specification as if it were individually recited herein.

This Background of the Invention section is intended to introduce various aspects of the art, which may be associated with embodiments of the present inventions. Thus, the forgoing discussion in this section provides a framework for better understanding the present inventions, and is not to be viewed as an admission of prior art.

### SUMMARY

There has been a long-standing and unfulfilled need for new and innovative drugs, medical products and imaging agents to address conditions of animals, mammals and humans. In particular, this long-standing and unfulfilled need is present in cancer diagnoses and treatments, other conditions of mammals and humans, and in the use of heavy metals in MRI imaging agents.

The present inventions, among other things, solve these needs by providing the compositions, materials, articles of manufacture, devices, methods and processes taught, disclosed and claimed herein.

There is provided a composition having therapy, imaging, diagnostic or theranostic applications, the composition having: a plurality of nanoparticles, wherein the nanoparticles comprise a backbone material; an active agent attached to the backbone; and, wherein, the plurality of nanoparticles has a predetermined particle size distribution defined by a D10=n−5, D50=n, D90=x+5.

Moreover, there is provided these nanoconstructs, nanoparticles, agents, compositions, methods, and devices having one or more of the following features: wherein n is a number in the range of about 5 nm to about 25 nm; wherein n is a number in the range of 7 nm to 22 nm; wherein n is a number in the range of about 10 nm to about 20 nm; wherein n is a number in the range of about 11 nm to about 15 nm; wherein the active agent is a photosensitizer; wherein the active agent is a photoacoustic agent; wherein the active agent is a sonosensitizer; having a second active agent; having a second active agent, wherein the second active agent is different from the active agent; wherein the active agent is selected from the group consisting of methylene blue, chlorin e6 (Ce6), coomassie blue, and gold; wherein the active agent is a terapyrroles; wherein the active agent is selected from the group consisting of a porphyrin, a chlorin, phthalocyanine, and a bacteriochlorin; wherein the active agent is selected from the group consisting of a HPPH, TOOKAD, LUZ 11, and BC19porphyrin; wherein the active agent is selected from the group consisting of a phenothiazinium salt, a benzophenothiazinium salt, a halogenated xanthene, squaraine; wherein the active agent is selected from the group of dyes consisting of methylene blue, toluidine blue O, PP9004, EtNBS, Rose Bengal, ASQI, Zinc(II) dipicolylamine di-iodo-BODIPY, and BIMPy-BODIPY; wherein the active agent is a transition metal co-ordination compound; wherein the active agent is a transition metal co-ordination compound having a metal selected from the group consisting of ruthenium, rhodium, platinum, gold and iridium, zinc, copper, and palladium; wherein the nanoparticles are 8PEGA; wherein the nanoparticles are BiPEG; wherein the nanoparticles comprise a targeting agent; wherein the nanoparticles comprise a targeting agent, wherein the targeting agent is F3-cys; and, wherein the nanoparticles are 8PEGA, wherein the active agent is Ce6, and wherein the nanoparticles comprise a targeting agent, wherein the targeting agent is F3-cys.

Further, there is provided a composition having therapy, imaging, diagnostic and theranostic applications, the composition having: a plurality of nanoparticles, wherein the nanoparticles comprise a backbone material consisting of PEG; an active agent attached to the backbone, thereby defining a plurality of nanoconstructs; wherein, the plurality of nanoconstructs has a narrow particle size distribution defined by a D10=n−5, D50=n, D90=x+5; and, wherein the plurality of nanoconstructs is capable of performing therapy, imaging, diagnostic and theranostic applications.

Still additionally there is provided a composition for use in destroying tumor cells, the composition having: an excipient, having a plurality of nanoparticles; a photosensitizer associated with the excipient; wherein, the excipient has a backbone consisting essentially of PEG; and, wherein the excipient has a particle size distribution defined by a D10=n−5, D50=n, D90=x+5.

Moreover, there is provided these nanoconstructs, nanoparticles, agents, compositions, methods, and devices having one or more of the following features: wherein, n is a number in the range of about 5 nm to about 25 nm, wherein the nanoparticles are 8PEGA, and wherein the active agent is Ce6; having a targeting agent; and, wherein the targeting agent is F3-cys.

Yet further there is provided a method of obtaining data for use in guiding therapeutic applications, the method having the steps of: administering an imaging agent having a plurality of nanoparticles to a subject; the nanoparticles being free from heavy metals; and, performing a nuclear magnetic resonance scan of the subject after administration of the imaging agent; wherein the nanoparticles are directly imaged; thereby providing an MRI of the nanoparticles and data related to the nanoparticles and the subject.

Further, there is provided these nanoconstructs, nanoparticles, agents, compositions, methods, and devices having one or more of the following features: wherein the nanoparticles comprise PEG; wherein the nanoparticles comprise 8PEGA; wherein the nanoparticles define a theranostic nanoconstruct; wherein the nanoparticles define a PDT nanoconstruct; wherein the data identifies the shape and position of a tumor; further using the data, at least in part, to provide a PDT; further using the data, at least in part, to provide a PDT; and obtaining an MRI of the nanoparticles after the PDT is provided; further having providing the data to a PDT system; and, further having providing the data to a medical record.

Still additionally, there is provided a method of providing a PDT, the method having: obtaining data from an MRI of nanoparticles in a subject; and, using the data, at least in part, to provide a PDT; wherein the nanoparticles are essential free from heavy metals.

In addition, there is provided these nanoconstructs, nanoparticles, agents, compositions, methods, and devices having one or more of the following features: wherein the nanoparticles have less than 1 ppm heavy metals; wherein the nanoparticles have less than 0.1 ppm heavy metals; wherein the nanoparticles have less than 0.01 ppm heavy metals; wherein the nanoparticles have less than 0.001 ppm heavy metals.

Furthermore, there is provided a method of providing a PDT, the method having: obtaining data from an MRI of nanoparticles in a subject; and, using the data, at least in part, to provide a PDT; wherein the nanoparticles are essential free from gadolinium.

Yet further, there is provided these nanoconstructs, nanoparticles, agents, compositions, methods, and devices having one or more of the following features: wherein the nanoparticles have less than 1 ppm heavy metals; wherein the nanoparticles have less than 1 ppm gadolinium; wherein the nanoparticles have less than 0.1 ppm gadolinium; wherein the nanoparticles have less than 0.01 ppm gadolinium; wherein the nanoparticles have less than 0.001 ppm gadolinium.

Moreover, there is provided a method of obtaining data for use in guiding therapeutic applications, the method having: administering an imaging agent having a plurality of nanoparticles to a subject; the nanoparticles being essentially free from gadolinium; and, performing a nuclear magnetic resonance scan of the subject after administration of the imaging agent; wherein the nanoparticles are directly imaged; thereby providing an MRI of the nanoparticles and data related to the nanoparticles and the subject.

Furthermore, there is provided a method of developing a PDT, the method having: obtaining data from an MRI of nanoparticles in a subject; and, using the data, at least in part, to develop a PDT; wherein the nanoparticles are essential free from heavy metals.

Still additionally, there is provided these nanoconstructs, nanoparticles, agents, compositions, methods, and devices having one or more of the following features: wherein the development of the PDT has an evaluation of a photosensitizer; wherein the development of the PDT has an evaluation of a targeting agent; wherein the development of the PDT has an evaluation of a nanoconstruct; wherein the data has a direct NMR image of the nanoparticles; and, wherein the subject is selected from the group consisting of animals, mammals and humans.

Furthermore, there is provided a method of developing a therapy, the method having: obtaining data from an MRI of nanoparticles; and, using the data, at least in part, to develop a therapy; wherein the nanoparticles have less than 1 ppm gadolinium.

Still additionally, there is provided these nanoconstructs, nanoparticles, agents, compositions, methods, and devices having one or more of the following features: wherein the development of the therapy has an evaluation selected from the group consisting of drug development, cancer treatment development, cardiac condition development, genetic material analysis, reaction pathway analysis and pharmacology; wherein the nanoparticles are imaged in vivo; and, wherein the nanoparticles are imaged in vitro.

Moreover, there is provided a method of developing a material, including: obtaining data from an MRI of nanoparticles; and, using the data, at least in part, to develop a material; wherein the nanoparticles have less than 1 ppm gadolinium.

Further, there is provided a method of evaluating a subject, the method including: obtaining data from an MRI of nanoparticles; and, using the data, at least in part, to evaluate a subject; wherein the nanoparticles have less than 1 ppm gadolinium.

Still additionally, there is provided these nanoconstructs, nanoparticles, agents, compositions, methods, and devices having one or more of the following features: wherein the subject is selected from the group consisting of a material, a drug, a process, a reaction pathway and a method of manufacturing.

Further, there is provided a nuclear magnetic resonance imaging agent, the imaging agent having: a plurality of nanoparticles that are essentially free from heavy metals; the nanoparticles having PEG; wherein the nanoparticles are capable of being directly imaged by a magnetic field generated by a magnetic resonance imaging system.

Moreover, there is provided a nuclear magnetic resonance imaging agent, the imaging agent having: a plurality of nanoparticles, wherein the nanoparticles have PEG; wherein the nanoparticles are capable of being directly imaged by the static, gradient, and radio frequency (RF) magnetic fields generated by a magnetic resonance imaging system, and thereby generate an image of the nanoparticles; and, wherein the imaging agent is essentially free from heavy metals.

Still further there is provided a nuclear magnetic resonance imaging agent, the imaging agent having: a plurality of nanoparticles that have less than 1 ppm gadolinium; the nanoparticles having PEG; wherein the nanoparticles are capable of being directly imaged by a magnetic field generated by a magnetic resonance imaging system.

Yet further there is provided a nuclear magnetic resonance imaging agent, the imaging agent having: a plurality of nanoparticles, wherein the nanoparticles having PEG; wherein the nanoparticles are capable of being directly imaged by a magnetic field generated by a magnetic resonance imaging system, and thereby generate an image of the nanoparticles; and, wherein the imaging agent has less than 1 ppm gadolinium.

Still additionally, there is provided an imaging agent having nanoparticles that are capable of being directly imaged by the magnetic field in a magnetic resonance imaging device, the nanoparticles having: a nanoconstruct having a backbone material, wherein the backbone material is non-paramagnetic; and, the nanoconstruct is capable of being directly imaged by a magnetic field.

Still additionally, there is provided these nanoconstructs, nanoparticles, agents, compositions, methods, and devices having one or more of the following features: wherein the nanoconstruct has about 3,600 protons; and, wherein the nanoconstruct is less than 25 nm; wherein the nanoconstruct has a photosensitizer; wherein the nanoconstruct has a targeting agent; wherein the nanoconstruct has a targeting agent and an imaging agent; and wherein the nanoconstruct is tumor avid.

Yet further, there is provided a method of performing a therapy in an MRI while directly obtaining images at least one of these imaging agents or these nanoparticles.

Still additionally, there is provided these nanoconstructs, nanoparticles, agents, compositions, methods, and devices having one or more of the following features: wherein the therapy has a surgery; and wherein the therapy has a PDT.

In an embodiment MRI imaging of nanoparticles with PEG is accomplished by using conventional MR pulse sequence with components added to selectively visualize the nanoparticle MRI signal and suppress other proton signals arising from water or fat. These sequences include but are not limited to spin-echo imaging methods, gradient-echo imaging methods, stimulated-echo imaging methods, echo planer imaging (EPI) methods, spiral imaging methods, back-projection imaging methods, and chemical shift imaging (CSI) or voxel-based spectroscopy methods.

In an embodiment there is provided the filtering out of other singles. To isolate the PEG nanoparticle signal from other proton signals, certain filtering components will be added to the pulse sequence of choice. These MRI signal filtering methods include but are not limited to pulse sequences with magnetic field gradient b values greater than 1,000 s/mm² to preferentially reduce tissue water MRI signal, conventional fat suppression schemes using radio frequency (RF) fat suppression or T₁ fat suppression, pulse sequences with sufficiently long TE times to take advantage of PEG proton long T₂ times and reduce both tissue water and tissue fat signals, pulse sequences based on or using the specific chemical shift of PEG protons, pulse sequences deployed with fat suppression and water suppression as indicated earlier and then using Dixon type imaging sequences to generate separate water and PEG proton images, pulse sequence using magnetization transfer (MT) to further suppress water in tissue and not suppress PEG protons, CSI pulse sequences to produce low resolution 1D, 2D, or 3D images of protons at the specific chemical shifts of water, fat and PEG protons, single voxel or multivoxel localized spectroscopy pulse sequences employing water and fat suppression methods to generate voxel NMR spectra

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an illustration showing the difference in structural representation of an embodiment of Ce6 delivery and ROS production efficacy (not drawn to scale), with the left illustration showing how ROS is produced by Ce6 in isolation, and the right showing an embodiment of an encapsulated Ce6 vs an embodiment of an anchored to 8PEGA in accordance with the present inventions. This difference in how ROS move shows a clear increase in efficacy.

FIG. 2 is an illustration showing an embodiment of the k-value plot of Ce6 encapsulated in PAAm NP as tracked by ADPA fluorescence quenching over time. The 660 nm OD=0.12 in PBS; the slope of the plot is the k-value.

FIG. 3A is an illustration of an embodiment of a modification of 8PEGA-Ce6 with F3-cys peptide, in accordance with the present inventions.

FIG. 3B is an illustration of an embodiment of an UV/VIS spectrum of 8PEGA-Ce6 and F3-8PEGA-Ce6 in PBS at 0.1 mg/m, in accordance with the present inventions.

FIG. 4 is an graph of an embodiment of a Hemocytometry Cell Population Results. Incubation conditions: 200 ug/mL F3-8PEGA-Ce6 for 24 hours in wells seeded with 200,000 cells; N=3×3 for control and test groups (3 plates each, tested 3 times each). Control group of cells contain no F3-8PEGA-Ce6 nanoconstructs, under the same conditions. The results show near identical cell populations, in accordance with the present inventions.

FIGS. 5A to 5E are photos of PDT testing images of HeLa 229 cells. FIG. 5A) Calcein AM fluorescence of PDT control cells (no F3-8PEGA-Ce6). FIG. 5B) Calcein AM fluorescence of PDT control cells 2 hours after illumination. FIG. 5C) Calcein AM fluorescence of test cells (with F3-8PEGA-Ce6) prior to PDT. FIG. 5D) Calcein AM fluorescence of test cells 2 hours after PDT. FIG. 5E) fluorescence 2 hours after PDT. PDT test plates were incubated with 200 ug/mL F3-8PEGA-Ce6 for 2 hours prior to PDT and all cells illuminated at a total fluence of 50 mW/cm² for 10 minutes, using a 692+/−20 nm filter and arc lamp. In accordance with the present inventions.

FIG. 6 is an image of diffusion-weighted spin-echo MR images of an embodiment of 8PEGA obtained with b=10⁸ s/m² (A) and b=10¹⁰ s/m² (B). The 110 M water proton signal dominates conventional MR images as seen in (A) but is suppressed by a factor of 10⁻¹⁰ by imaging at b=10¹⁰ s/m². The diffusion constant of 8PEGA was measured by stimulated echo pulsed field gradient NMR at 25° C. to be 3.572 10⁻¹¹ m²/s, allowing 70% of the initial magnetization to survive at b=10¹⁰ s/m². The color bar in (B) shows detected concentration of 8PEGA, in accordance with the present inventions.

FIG. 7 is a graph of a concentration dependent MRI signal of an embodiment of 8PEGA. A region of interest (ROI) was selected for each of the 5 vials and mean (circles) and standard deviation (error bars) of the signal computed. A linear equation was fitted to the 5 measured vials and the result is shown as a dashed line, in accordance with the present inventions.

FIG. 8 is a graph illustrating size distribution of embodiments of nanocomposites in accordance with the present inventions.

FIG. 9 is a schematic illustrating an embodiment of a PDT in accordance with the present inventions.

FIG. 10 is a chart illustrating an example embodiment of an 8-arm-PEG-amine (8PEGA) in accordance with the present inventions.

FIGS. 11-18 are charts showing performance and nature of embodiments in accordance with the present inventions.

FIG. 19 is a graph showing an embodiment of a K-value plot of PAAm-Ce6 NPs at OD equal to that used in 8PEGA-Ce6 experiment, in accordance with the present inventions.

FIG. 20 are spectrum of embodiments of Ce6 on 8PEGA in accordance with the present inventions, illustrating that addition of Ce6 to 8PEGA, with or without targeting, does not cause aggregation, fluorescence is maintained, and it still produces ROS.

FIG. 21 is an embodiment of a graph showing the change in signal from 8PEGA as a function of applied gradient (b-value); the slope gives the diffusion constant D, in accordance with the present inventions.

FIG. 22 are images showing the selective uptake of CTP targeted 8PEGA-Ce6 in myocytes. Ce6 fluorescence is found only in myocytes.

FIG. 23 is a TEM of an embodiment of 8PEGA in accordance with the present invention. 8PEGA is a hydrogel and TEM uses a vacuum for measurement, so the grids were inserted while still wet with potential that some 8PEGA would remain in the hydrated confirmation due to surface tension on the plate. Avg range is ˜10-12 nm.

FIG. 24 are images showing that an embodiment of PDT with targeted 8PEGA NP does not cause damage to vasculature, in accordance with the present inventions. PI fluorescence is only from myocytes.

FIG. 25 are images showing an embodiment of a non-targeted PDT with Ce6, in accordance with the present inventions. PI fluorescence is no longer confined to myocytes and appears in coronary vessel cells, showing vasculature damage.

FIG. 26 is a perspective schematic diagram of an embodiment of an MRI and a process for using an MRI in accordance with the present inventions.

### DETAILED DESCRIPTION

In general, the present inventions relate to nanoconstructs, methods of making these nanoconstructs, and therapy, imaging, diagnostic, theranostic and other applications for these nanoconstructs.

Generally, in an embodiment, a nanoconstruct (NC) is capable of simultaneously serving as a therapeutic platform, as well as, an imaging agent. These nanoconstructs may also have other active components, providing other capabilities, such as for example targeting agents to select a specific cell type, specific structure, or have specific membrane related properties, such as cellular membrane permeabilities.

Generally, preferred embodiment is a multi-functional, ultra-small, nanoplatform that has a plethora of desirable therapeutic, imaging, diagnostic, theranostic, and combination and variations of these properties. This preferred embodiment has superior photodynamic efficacy, compared to photosensitizers alone, and other PDT nanoconstructs. In a utilization, this preferred nanoconstruct has superior photodynamic efficacy in its application to cancer cells. In addition to its superior efficacy, this NC is non-toxic, and is a molecular imaging agent for MRI.

8-arm polyethylene glycol amine (8PEGA) is a biocompatible polymer that allows for a range of modifications. Typically, the amine groups may be used for covalent anchoring of a range of photosensitizers (PSs) in photodynamic therapy (PDT). Additionally, in embodiments, other arms of the polymer may then be converted to maleimide groups, permitting the attachment of various cysteine terminated peptides. For example, peptides can have an extra amino acid attached (a cysteine) to get that free thiol that is then utilized. In such an embodiment there is a peptide+1 amino acid, where the peptide function is retained. Embodiments of 8PEGA as an NC that may be flexibly tailored to target and apply PDT in a host of biological environments, such as cancer, heart arrhythmia, and choroidal neovascularization, to name a few. 8PEGA also possesses a long T₂ lifetime for MRI, and is used as an imaging agent, for example, in vivo through conventional diffusion weight imaging (DWI) at high b-values, where the water signals may be sufficiently suppressed by a combination of methods to yield clean, direct images.

The present NCs, systems and methods, provide many improvements over prior therapies, imaging systems, prior nanoparticles (NPs), and treatments. These improvements include for example: optimized reactive oxygen species (ROS) production; small size allowing for penetration of any desired biological area; ease of modification enabling the varied biological system targeting and NC accumulation; increased bio-elimination rate owed to the small size and bio-degradability of 8PEGA; and, improved DWI over conventional methods.

In an embodiment for use as an NMR imaging or contrast agent, in an embodiment of the NCs, typically b values between 10⁸ and 10⁹ s/m² are used in DWI, but the long T₂ lifetime of 8PEGA and high MW (40 kDa) allows for use up to b=10¹⁰ s/m², leading to sufficient suppression of water/fat signals, leaving only 8PEGA. Additionally, the use of 8PEGA in DWI would allow for MRI without the need for heavy metal atoms that consistently raise safety concerns.

These nanoconstructs can be formulated into a drug delivery system, such as for: delivery directly into the blood stream, e.g., prepackaged IV formulation or disposable prepackaged syringe; ingestion, such as a pill, tablet, or liquid; inhalation, such as through metered dose inhalers or nebulizers; topically, such as ointments or liquids for transdermal deliver or in vivo, as part of an insufflation gas. These drug delivery systems can have one, two, three or more different nanoconstructs, which each being specifically designed, or specifically purposed. These drug delivery systems may also contain other additives and active agents, that are not nanoconstructs, and which function as, for example, additional imaging and therapeutic agents.

Generally, the additives can be associated with the nanoconstruct backbone, e.g., the nanoparticle, by way of: chemical bonds (e.g., covalent, ionic, Van der Waals); sterically or mechanically, such as through steric hinderance or physical capture within or by the backbone; they can be a part of the molecular structure that makes up the backbone material; and combinations and variations of these. The additives can be added prior to the formation of the nanoparticle, during the formation of the nanoparticle, after the formation of the nanoparticle, and combinations and variations of these.

Many imaging dyes or agents, and other diagnostic tools have used materials that may be viewed as undesirable, especially for use with humans. These dyes and agents may be metal based, and use or contain metals, metal oxides, or metal compounds or complexes, such as iron, iron-platinum, magnesium and manganese. MRI imaging agents use gadolinium based materials.

Embodiments of the present nanoconstructs that are free from heavy metals, e.g., the nanoconstruct, the drug delivery system for the nanoconstruct and both contain less than about 10 ppm heavy metals, less than about 1 ppm heavy metals, and less than about 0.1 ppm heavy metals, and zero heavy metals. Heavy metals would include titanium and all heavier metals. These heavy metal free nanoconstructs are capable of function as imaging and diagnostic agents. An embodiment of these imaging nanoconstructs provides an MRI imaging agent that is gadolinium free, e.g., having less than 0.1 ppm, and preferable zero gadolinium. The heavy metal free NPs, NCs, drug delivery systems and combinations and variations of these, are capable of being directly imaged by MRI and thus functioning as a direct MRI imaging agent, diagnostic agent and both.

An embodiment of a targetable NC is capable of simultaneously serving as a therapeutic platform for photodynamic therapy (PDT), as well as an MR molecular imaging agent. In an embodiment this nanoconstruct is free of heavy metal atoms, and in particular gadolinium.

In an embodiment an ultra-small 8-arm polyethylene glycol amine (8PEGA) NC, with an attached chlorin e6 (Ce6) PS, and CTP-cys (cardiac targeting peptide) targeting moiety, yield therapeutic results for PDT of heart arrhythmia, in vivo and ex-vivo on live rat and sheep hearts, respectably, when using targeting peptides for cell-specific destruction of cardio-myocytes. This NC was the octopus-like, ultra-compact and highly biocompatible polymer, 8-arm polyethylene glycol amine (8PEGA). The amine terminated arms anchored the algae derived PS, chlorin e6 (Ce6), and a targeting moiety for cardio-myocytes. This nanoconstruct can be used as an MRI imaging agent and used as an MRI theranostic for the cardiac conditions as well as other conditions.

In an embodiment this nanoconstruct can be configured such that it provides PDT to cancer. For this purpose, the targeting peptide can be F3-cys. The 8PEGA-Ce6 NCs have a superior reactive oxygen species (ROS) production compared to traditional Ce6 encapsulated Polyacrylamide (PAAm) NCs. This provides, among other things, and in some applications, benefits in PDT for 8PEGA NC when compared to PAAm NCs.

The superior reactive oxygen species proved by the 8PEGA-Ce6 NC is singlet oxygen, as Ce6 produces singlet oxygen. The production is defined as superior such that effectively more of what is produced is useable in oxidative stress by being more widely available in the cells, compared to NP encapsulation where ROS may not always escape the matrix of the NC. In addition, NCs like 8PEGA or PAA act as effective tools in preventing aggregation of a PS, which the free PS may do, and thus decease production due to quenching of excited states. In making this comparison, it is preferable that, k-values of Ce6-8PEGA and Ce6-PAA are compared at identical optical densities.

The 8PEGA-Ce6 NC is also cyto-compatible and offers chemical flexibility for the attachment of a choice of targeting peptides. Finally, this label-free 8PEGA NC can be directly and selectively imaged by MRI, using standard spin-echo imaging sequences with large diffusion magnetic field gradients to suppress the water signal. Notably, due to its ultra-small size this NC has improved in vivo penetration and bio-elimination.

Generally, it is theorized that any peptide is viable for attachment to PEG nanoparticles, and it is believed that all peptides that are cysteine terminated can be attached to 8PEGA. The F3-cys peptide is a specific cancer targeting peptide. In an embodiment it was used for the application of 8PEGA-Ce6 to cancer. HeLa cells were chosen as the model system of interest due to their robust nature and known over-expression of nucleolin, the specific target of the F3-cys peptide. In addition to advantages from 8PEGA's small size, uniformity, and biocompatibility, further advantages have been developed by the present inventions, these include; optimized ROS production with a given PS, and is used as a molecular imaging agent for MRI. It is theorized that the optimized ROS production is expected for this NC due to, for example, the direct contact of the PS Ce6 with the oxygenated environment, in contrast to when it is encapsulated inside a standard model matrix, such as in Polyacrylamide hydrogel nanoparticles (PAAm NPs). In this manner, in embodiments there is provided a nanoconstruct that has increased ROS production efficacy, biocompatibility, and flexibility in targeting when compared to prior NCs.

In embodiments of the present inventions 8PEGA based nanoparticles, and nanoconstructs, are used as an MRI imaging agent, for imaging applications, diagnostic applications, theranostic applications, and combinations and variations of these. These 8PEGA MRI imaging agents can be used in conjunction with other therapies, imaging or contrast agents, and applications. It is theorized that the high molecular weight (e.g., 40 kDa), flexible chain dynamics of embodiments of the 8PEGA group, and its specific structure, in part, create the favorable conditions for the present invention's highly selective molecular imaging MRI agent. Further, the slow diffusion constant and transverse spin relaxation rate of 8PEGA combine to allow diffusion weighted MRI sequences which suppress surrounding water signals, providing a clean image of 8PEGA. In embodiments of the 8PEGA MRI imaging agent and the 8PEGA MRI imaging applications of the present inventions, the 8PEGA MR signal is selectively detected and is proportional to its concentration. The 8PEGA MRI imaging agent and applications is superior to current MRI imaging agents and applications, because of among other things, its biocompatibility, compared to current heavy metal atom MRI imaging agents. Further, the 8PEGA MRI imaging agent is also a theranostic agent or has theranostic capabilities providing added functionality and benefits over current MRI imaging agents and applications.

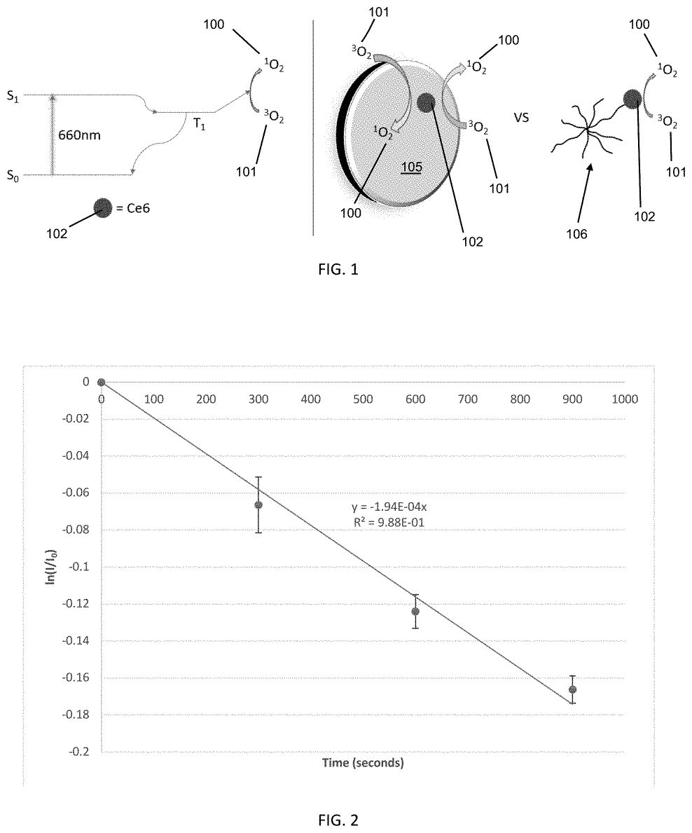

ROS production of Ce6 when attached to 8PEGA vs. when encapsulated in polyacrylamide (PAAm), is compared where the two competing nanostructures are represented in FIG. 1. FIG. 1 shows the production of ROS 100, excited singlet state (¹O₂) 100 from its triplet ground state 101 of molecular oxygen (³O₂) when Ce6 102 is exposed to light having a wavelength of 660 nm. The production of ROS 100, and the path for the ROS, when the Ce6 102 is encapsulated within a PAAm NC (Ce6/PAAm NP) 105 is compared to the production of ROS 100 when the Ce6 102 is part of an 8PEGA NC (8PEGA-Ce6) 106.

FIG. 2 is a graph of an embodiment of the “k-value” plot of the relative ROS production of PAAm encapsulated Ce6. The k-value is a measure of the kinetic rate at which ROS is produced by Ce6, as measured by the first order decay of ADPA fluorescence. To generate k-values that are comparable, the Optical Density (OD) of the PAAm-Ce6 NPs was adjusted by UV/VIS to the OD (0.12) of 8PEGA-Ce6 at 660 nm. Table 1 shows the relative results for the two NCs when normalized for their literature ODs.

TABLE 1
Table 1: k-values of the two discussed NCs at OD = 0.12 for 660 nm.

| Nanoplatform | OD | k-value |
|---|---|---|
| 8PEGA-Ce6 | 0.12 | 2.99E−04 |
| | | s⁻¹ |
| Ce6/PAAm | 0.12 | 1.94E−04 |
| NP | | s⁻¹ |

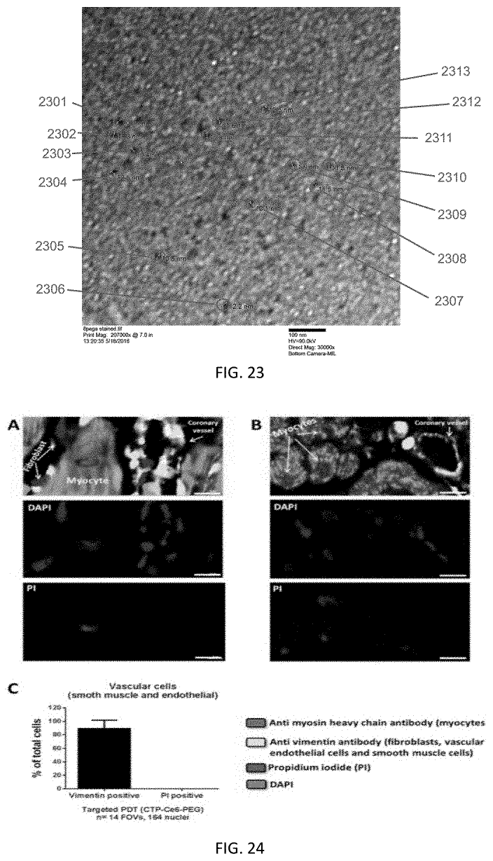

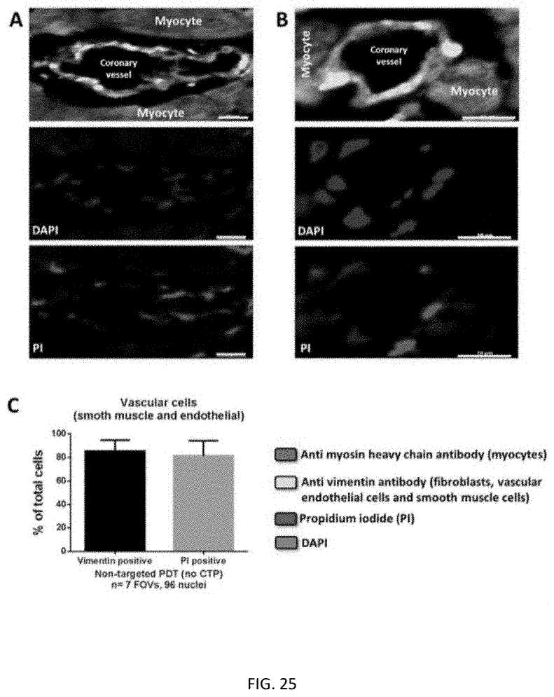

The diameter of the NC 8PEGA is also calculated by Stokes-Einstein approx. and by TEM, as shown in FIG. 23.

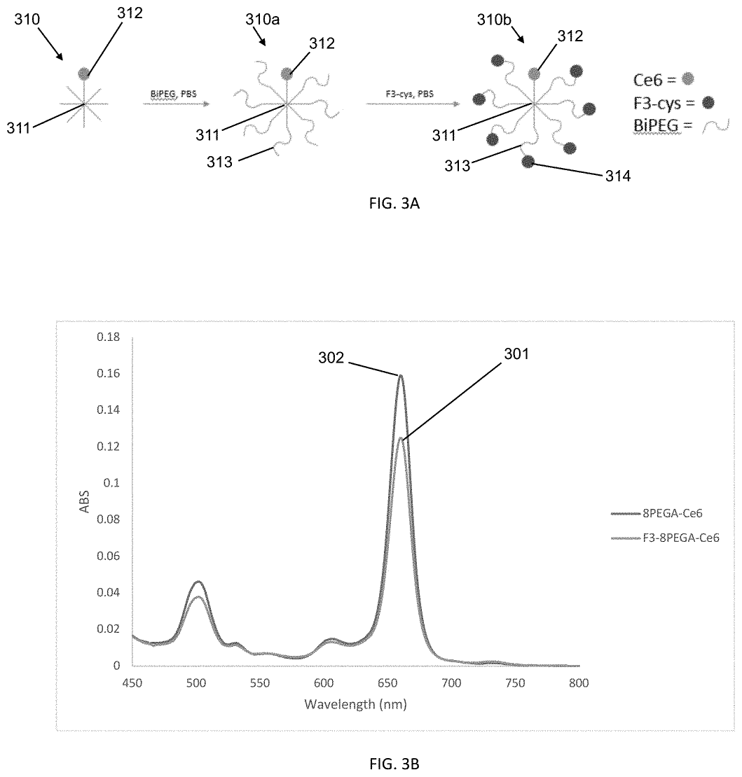

FIG. 3A shows the absorption spectrum of the F3-8PEGA-Ce6 conjugate 301, which is a targeted NC, and 8PEGA-Ce6 conjugate 302, which is a non-targeted NC. The characteristic peaks at 660 nm are preserved for both NCs.

FIG. 3B provides an illustration showing the chemical modification of 8PEGA-Ce6 with F3-cys. Thus, an NC 310 having 8PEGA 311 and Ce6 312, is modified with BiPEG, e.g., 313, to provide NC 310a, which is then modified with F3-cys, e.g., 314 to provide targeted NC 310b.

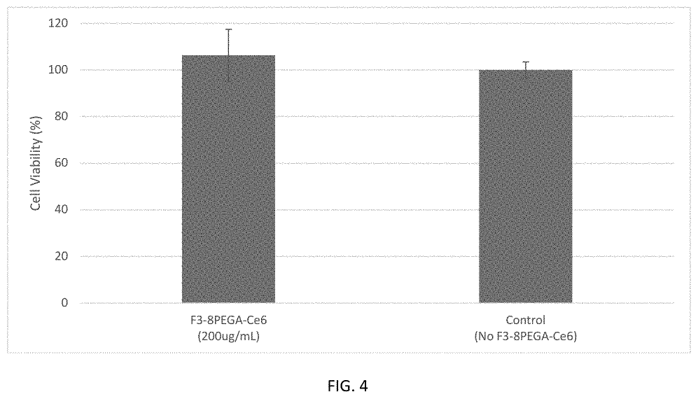

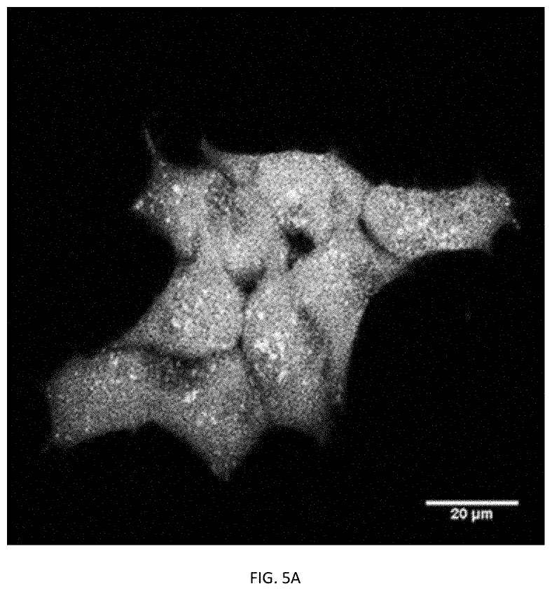

Flow cytometry was employed as a method of testing the biocompatibility of this F3-cys-8PEGA-Ce6 NC to test for dark toxicity. Cells were tested at a concentration of 200 ug/mL F3-8PEGA-Ce6; control cells denote a data group with no F3-8PEGA-Ce6 NCs added. As can be seen from FIG. 4, no significant toxicity was observed.

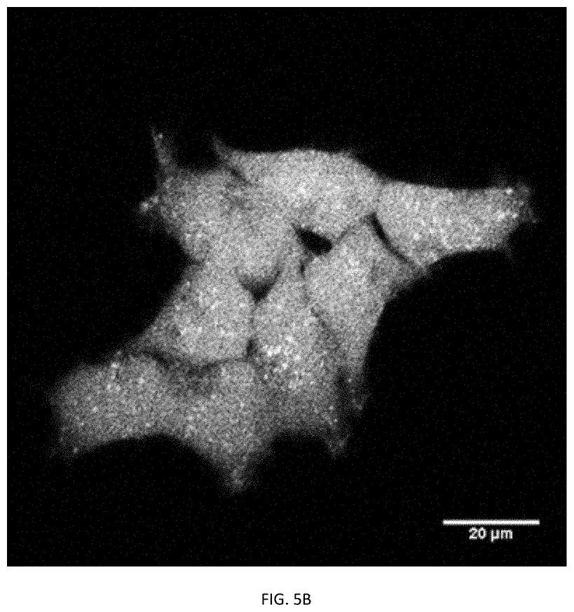

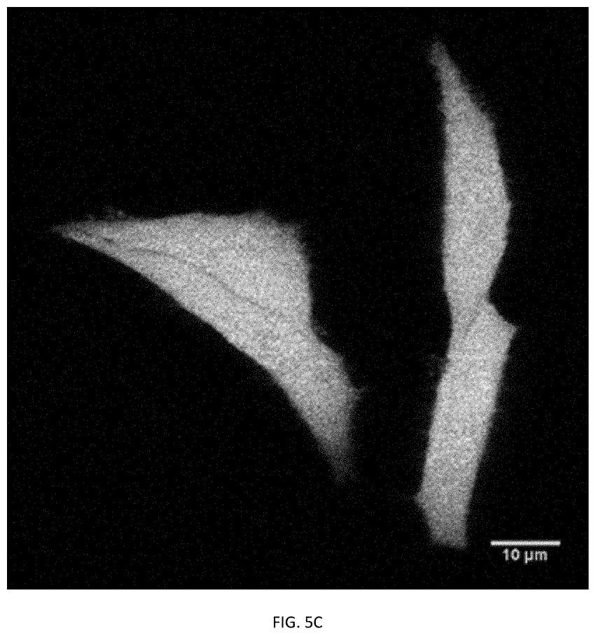

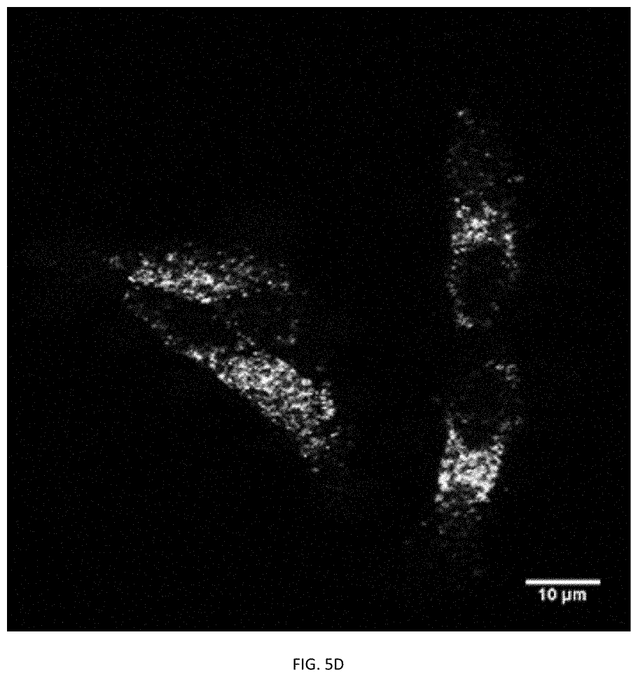

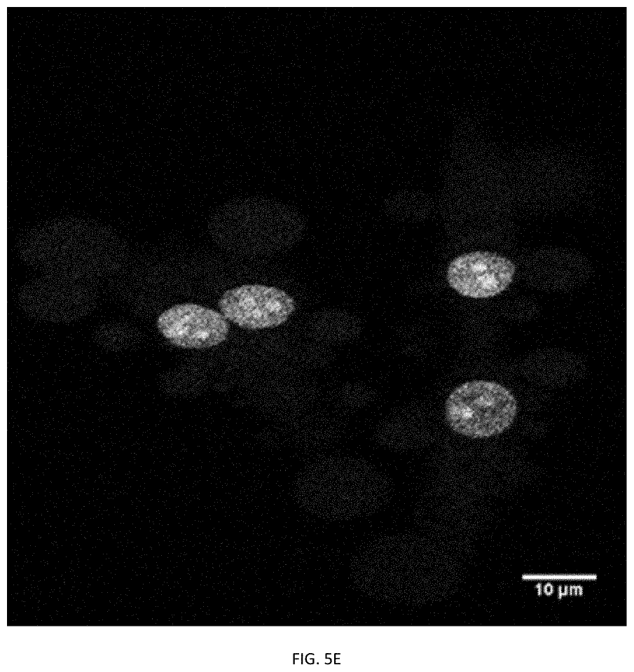

As a control test to eliminate the possibility of simple cell stress from the excitation light, HeLa cells without F3-8PEGA-Ce6 were plated and illuminated for the same length of time and power as used in PDT for NC-treated cells (50 mW/cm², 10 min). After illumination (as shown in FIG. 5A, B) there is an insignificant change in cell morphology, no change in the cytosolic stain calcein AM fluorescence counts, and no signs of membrane blebbing (a hallmark of apoptosis).

A remarkable difference is seen in the calcein AM fluorescence after photo-illumination of the cells with F3-8PEGA-Ce6 (as shown in FIG. 5C, D). While there was no observable propidium iodide (PI) fluorescence prior to illumination (data not shown), after illumination cell membrane impermeable PI can be seen to stain the nuclei of the cells (as shown in FIG. 5E).

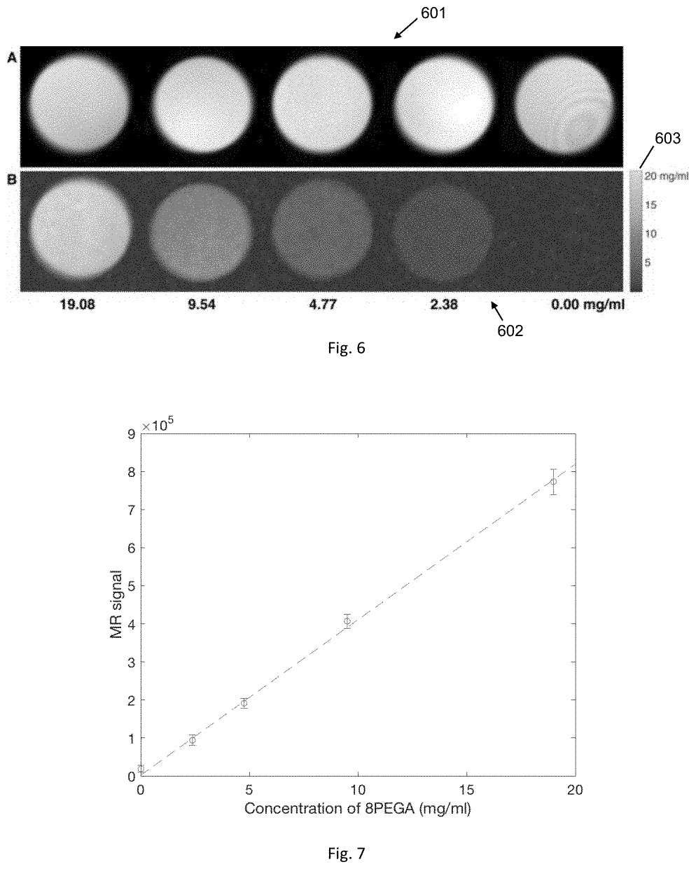

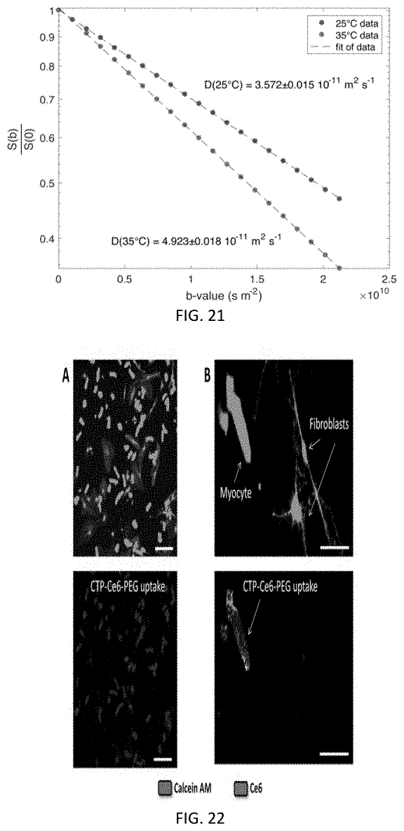

The translational diffusion constant, D, and relaxation times T₁ and T₂ of 8PEGA were measured at 25 and 35° C. (as shown in Table 2, FIG. 21). Images in solution of 8PEGA (non-modified) were gathered to demonstrate generation of clean images when water/fat suppressed (as shown in FIG. 6) and their concentration dependent response (as shown in FIG. 7). Thus, FIG. 6 image 601 shows 8PEGA obtained with b=10⁸ s/m² and image 602 shows 8PEGA obtained with b=10¹⁰ s/m². The 110 M water proton signal dominates conventional MR images as seen in image 601; but is suppressed by a factor of 10⁻¹⁰ by imaging at b=10¹⁰ s/m². The bar 603 correlates to the detected concentration of 8PEGA in image 602. This response can be seen to be linear with concentration (as shown in FIG. 7), consistent with images gathered with water suppression techniques (as shown in FIG. 6B). Tested concentrations were 0, 2.38, 4.77, 9.54, and 19.08 mg/mL 8PEGA in H₂O. In addition, the Stokes-Einstein equation:

r=kT/6πηDₜᵣₐₙₛ

is employed to calculate the size of 8PEGA to verify TEM results; the diameter is calculated to be 10.96 nm at 35° C., consistent with the ˜10-12 nm range found in the 13 measured NCs (as shown in FIG. 23).

TABLE 2
Table 2. Relaxation times and translational diffusion
constant of 8PEGA at 25 and 35° C. measured at 16.4 T.
Sample is 5 mg/mIL8PEGA in 25/75 (V/V) H₂O/D₂O.

| T (° C.) | T₁ (ms) | T₂ (ms) | D (10⁻¹¹ m²/s) |
|---|---|---|---|
| 25 | 791 ± 36 | 586 ± 24 | 3.572 ± 0.015 |
| 35 | 934 ± 72 | 769 ± 31 | 4.923 ± 0.018 |

Ce6-8PEGA is a more efficient ROS producing platform, compared to hydrogel NPs, based on, among other factors, that in 8PEGA the Ce6 group is in direct contact with the oxygenated environment of the cells. This configuration is illustrated in FIG. 1. Thus, oxygen does not need to diffuse into a PAAm NP matrix 105 encapsulating the PS 102 and the ROS 101 need not diffuse out, nor suffer losses due to reaction with the matrix, among other things. This feature is confirmed by the k-value test by showing that, when adjusted to identical ODs, the k-value of the Ce6-8PEGA is about 50% larger than that of the Ce6 encapsulated PAAm NPs (Table 1).

Being that 8PEGA is a star shaped polymer, measurement of its size by the Stokes-Einstein Equation, which assumes a spherical material shape, is desirable. It is found after measuring the translational diffusion coefficient, D (Table 2, FIG. 21), that the size of polymer at 35° C. is ˜11 nm. As a secondary method of analysis, 8PEGA stained with uranyl acetate and visualized using TEM (as shown in FIG. 23). The size of the 13 chosen points, within circles 2301 (13.1 nm), 2302 (11.3 nm), 2303 (10.4 nm), 2304 (12.5 nm), 2305 (10.8 nm), 2306 (12.2 nm), 2307 (10.1 nm), 2308 (11.5 nm), 2309 (11.9 nm), 2310 (10.8 nm), 2311 (10.9 nm), 2312 (11.6 nm), 2313 (12.5 nm) is found to be approx. 10-12 nm, in good agreement with the Stokes-Einstein Equation measurement.

Unlike PEGylating the surface of nanoparticles, in the embodiments of the present inventions the nanoparticle backbone itself is made of primarily of PEG, e.g., at least about 85% PEG, at least 90% PEG, at least 95% PEG, at least 99% PEG, at least 99.9% PEG and preferably 100% PEG. Thus, a targeting vector is helpful not only for in vivo applications but even to accelerate cell uptake in vitro. For cancer targeting, for example, the nucleolin targeted peptide F3-cys was chosen for grafting onto the 8PEGA-Ce6. Notably, after attachment of F3-cys, the Ce6 spectroscopic features are largely unaffected, as shown in FIG. 3B, indicating the preservation of photophysical properties when switching peptides (from CTP for targeting heart myocytes to F3 for targeting cancer cells). A mild decrease in Ce6 absorption is noted for 0.1 mg/mL when compared to before and after modification, but is expected, as the BiPEG and F3-cys will increase the MW of the NC. BiPEG is a 2 arm bi-functional PEG (e.g., 2 kDa). Embodiments of BiPEG can have NHS ester on one end, and maleimide at the other end. In embodiments, this functions to convert the amines to maleimides for the peptides to be attached.

An important aspect of embodiments of the present NCs is their biocompatibility. In an embodiment, the total construction is comprised of PEG, Ce6, and the homing peptide F3-cys. PEG is a highly biocompatible substance and F3-cys is a good targeting agent, with no toxic effects in vitro or in vivo. The algae derived Ce6 is an example of a PDT agent. Embodiments of NC having these three moieties present no significant biocompatibility issues, e.g., the NCs are biocompatible. This is confirmed in vitro by hemocytometry results as shown in FIG. 4.

Before initiating PDT tests with F3-8PEGA-Ce6, the chosen laser conditions (50 mW/cm² for 10 min) were tested to ensure that the illumination was not a source of significant cell stress. Calcein AM images of the cells before (as shown in FIG. 5A) and after (as shown in FIG. 5B) illumination demonstrated little change in morphology and no signs of apoptosis. Therefore, the photo-illumination source does not impart significant stress upon the cells. PDT was then initiated in the presence of F3-8PEGA-Ce6 at a concentration of 200 ug/mL (as shown in FIGS. 5C/D/E). There is a significant decrease of calcein AM fluorescence after PDT, indicating a loss of cytosolic contents, an event that would only occur under conditions where the cell membrane has been ruptured. Rupturing of the membrane was shown by the staining of the nuclei with the cell impermeable dye PI (as shown in FIG. 5E). Taking the PDT test results in conjunction with the cyto-compatibility in FIG. 4, it is evident that the death of the cells is PDT-mediated.

In addition to the more efficient PDT (50% larger k-value), it is believed that the use of 8PEGA NCs, therapies and theranostics, present many additional advantages, including for example: (1) The small size of the NC offers the possibility of quick renal clearance from the body, a feature not afforded by larger NPs; and, (2) the ability to penetrate tumor areas that have not yet undergone angiogenesis, in contrast to traditional larger NPs that require a porous/leaky vasculature so as to be able to penetrate the tumor.

Prior to the present inventions, it was believed that PEG needed to be ¹³C tagged, before it could be selectively imaged in vivo using heteronuclear MR methods. Embodiments of the present PEG NPs and NCs exhibit the surprising capability to be an NMR, MRI, imaging agent, contrast agent, and combinations and variations of these. In the PEG embodiments, among others, the intrinsic flexibility of the polyethylene oxide chain and the slow translational diffusion of 8PEGA create an exploitable set of physical and dynamic conditions for selective MR imaging of 8PEGA protons using ¹HNMR. Specifically, 8PEGA's fast chain motions with correlation times of approximately 0.1 ns provide sufficient averaging of the proton dipole-dipole interaction to yield a long nuclear spin transverse relaxation time T₂, measured here to be 586 and 769 ms at 25 and 35° C., respectively. In contrast to fast internal chain dynamics, the molecule's high molecular weight yields a translational diffusion constant that is two orders of magnitude slower than that of water molecules. Therefore, the water signal can be effectively suppressed by large diffusion gradients so that only the ethylene oxide signal will remain due to the combination of its long T₂ time and slow diffusion if 8PEGA. Notably, the MR signal intensity decays as:

M_{xy}(b,TE)=M_{xy}(0)e^{-bD}e^{-TE/T}^{₂}

where the b value is determined by the magnetic field gradient magnitude and duration, D is the translational diffusion constant of either water or 8PEGA, TE is the echo time, and T₂ is the transverse spin relaxation time. In addition, the symmetry of the ethylene oxide monomer gives rise to a single chemical shift for all four protons and each 40 kDa polymer molecule caries approximately 3,600 protons, creating a large molar amplification of the NMR or MRI signal. By performing a diffusion-weighted, spin-echo MR imaging experiment with high b values and long TE times, water signals, due to fast diffusion, and fat signals, due to short T₂ times, are effectively suppressed and the 8PEGA signal selectively imaged. In vivo, a small portion of water signal intensity at high b values will remain due to restricted diffusion of water molecules in cells, but these signals can either be removed or distinguished from the 8PEGA signal due to the ˜1 ppm difference between water and ethylene glycol protons in traditional ¹HNMR. It is noted that the number of protons, depending upon the nanoparticle, can be greater or lesser than 3,600, can be from about 2,000 to about 30,000, greater than about 4,000, greater than about 5,000, about 2,000 to about 10,000, about 3,000 to about 7,000 and all values within these ranges, as well as greater and lesser amounts.

Thus, as seen for example in FIG. 6, 8PEGA functions very well as an MR imaging agent when coupled with the above-mentioned suppression techniques. This provides a significant advancement and the ability to replace or eliminate other MRI contrast agents, having less than advantageous, problematic or hazardous, materials, like gadolinium salts or chelates, which are the subject of health concerns, safety concerns, and present health risks to certain patient groups. There is a clear difference in images 601 without and images 602 with applied suppression techniques. The 8PEGA imaging signal is also linear with its concentration (as shown in FIG. 7). When the above embodiments of imaging agents and imaging techniques are applied, clean and well-defined images of 8PEGA are recovered, showing clearly its viability as an imaging agent in vivo. Under properly calibration, this provides for precise imaging and diagnoses, including allowing for quantification of the 8PEGA in biological tissue (e.g. tumor area vs filtration organs).

Thus, the 8PEGA-Ce6 NC provides an NC have one, or more, and preferably all of the following features: superior reactive oxygen species, MRI imaging capabilities, heavy metal free, and capable of having cancer targeting agents.

The ability to have a targeted nanoconstruct that is both an imaging agent and a PDT agent creates an efficacy in the treatment of conditions that was heretofore unheard of. This targeted theranostic nanoconstruct uses a targeting agent to target the specific structure, e.g., cell type, tumor, etc. In this manner the targeted theranostic nanostructure will selectively associate with the targeted structure by the action of the targeting agent. Targeting agents alone can provide good specificity, with about 80%, about 90% and about 95%, of the nanoconstructs being associated with the targeted structure. The targeting agent, however, cannot provide absolute specificity to the targeted structure. Thus, when the activation energy is delivered it is desirable to be able to image the target structure and thereby preferably determine a precise pattern for the delivery of the activation energy, e.g., the light.

In this manner, in an embodiment of a theranostic method, the targeted theranostic nanoconstruct is delivered to the body, and is carried by the blood and associates with the targeted structure, e.g., a tumor. An MRI imaging of the tumor is taken, and this image being enhanced by the presence of the nanoconstruct. The position and shape of the targeted structure in the body is obtained and stored. Subsequent image techniques, e.g., photo acoustic imaging, modeling techniques, e.g., computer enhancements and rendering of the initial MRI image, and both can be used to provide very precise image and position data and information for the targeted structure in the body. An illumination pattern can then be developed based upon this image and position data. This illumination pattern can be predetermined, customized and specific to the targeted structure.

In an embodiment this predetermine illumination pattern can be a small diameter laser spot. The energy of the laser beam delivered in this pattern is sufficient to activate the PS causing the production of ROS. The energy delivered by the laser beam, however, is below the threshold where laser induce optical break down of tissue occurs (LIOB), and preferably below the threshold where the tissue is heated. In embodiments the properties of the laser beam, e.g., wavelength, focal length, scan time or duration, power, pulsed, pulse length or continuous, and spot size can be determined so that the PS is activated in very precise locations, (z, x and y coordinates), down to a cellular and subcellular level; and with little to no damage to the targeted structure's tissue from direct interaction with the laser. In this manner the PS can be activated with cellular precision, and provide ROS to the targeted structure without damage to adjacent cells to that structure. (It being understood that ROS has a very limited duration after being created, and if created within or adjacent to cell, will likely not migrate to or effect non-adjacent cells.)

In embodiments of the nanoconstructs, both spatial (laser focused) and biological (cell selective) selectivity is achieved by employing nanoconstructs (NCs) with targeting antibodies or peptides, which also extended PDT treatment to subsurface tumors. In general, the use of NCs allows for protection of a PS from the bio-environment, and vice versa, for bypassing the immune system.

The relatively small sized (<20 nm) 8PEGA derived NCs penetrated the target tissue, including very dense tissue such as muscle, selectively accumulating in specific cell types, e.g., myocytes, and thus allowing their photodynamic destruction under mild near infrared illumination. In treating cancer, the small size of these NCs will provide a tumor avid NC that alone, or in conjunction with a targeting moiety, will be cell-selective for the tumor.

Any type of active dynamic therapy moiety can be used with a nanoparticle and preferably a targeting agent to form a nanoconstruct; and for example, a theranostic nanoconstruct. In an embodiment, any presently know, or later developed, dynamic therapy agent is combined with a nanoparticle that is formed at least in part from PEG, a PEG based material, and combinations and variation of these. In an embodiment, any presently know, or later developed, dynamic therapy agent is combined with a nanoparticle that is has a cross section of less than 50 nm, and embodiments of less than 40 nm. In an embodiment, any presently know, or later developed, dynamic therapy agent is combined with a nanoparticle that has a cross section from about 5 nm to about 20 nm, from about 5 nm to about 15 nm, from about 10 nm to about 15 nm, and from about from about 9 nm to about 12 nm. In an embodiment, any presently know, or later developed, dynamic therapy agent is combined with a nanoparticle that is has a cross section of less than 50 nm, less than 40, nm, less than 30 nm, less than 20 nm, less than 15 nm and less than 10 nm. In an embodiment, any presently know, or later developed, dynamic therapy agent is combined with a nanoparticle that is an 8PEGA. All of these embodiments of nanoconstructs may also have targeting agents, having targeting capability or features, e.g., tumor avid, and combinations and variations of these.

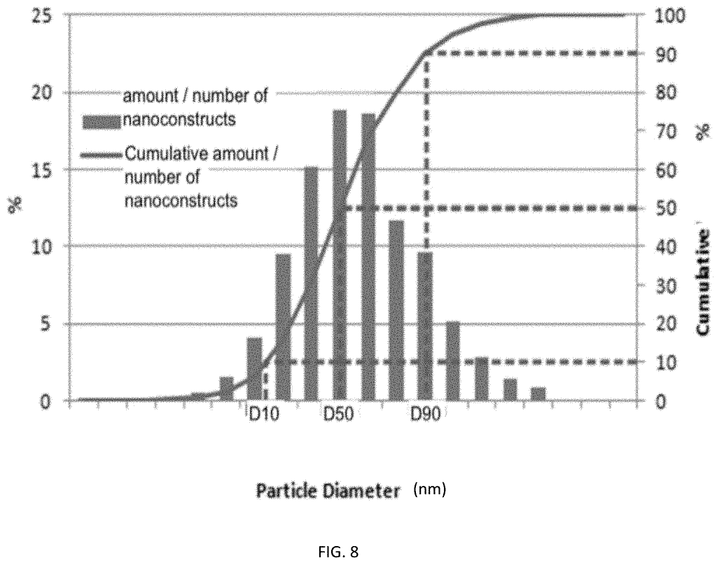

Combinations and variations of the above embodiments on nanoconstructs, and others taught in this specification, are used as, or are a part of, a drug product. In an embodiment of these drug products, the nanoconstructs have a uniform size, and a highly uniform size. Thus, in embodiments, the nanoconstructs in a drug product, and in particular a dosage of a drug product for use with a subject or patient (animal, mammal, or human) have particles that have a size difference of no greater than about 1%, no greater than about 5%, and no greater than about 10%. In embodiments, the nanoconstructs in a drug product can have a particle size distribution of: D10=n−5, D50=n, D90=x+5 (where n=5 to 25 nm); D10=n−10, D50=n, D90=x+10 (where n=5 to 25 nm); D50 of about 10, D50 of about 15 nm, D50 of about 20 nm, D50 of about 50 nm, D50 of from about 8 nm to about 15 nm, and larger and smaller values. (FIG. 8, illustrates the computation, and distributions, for the D10, D50 and D90 values. The D50 is that value that represents the size of nanoconstructs that make up 50% of the cumulative amount in a drug product. D-90 represents the size of nanoconstructs that makes up 90% of the cumulative amount in the drug product. D-10 is that value that represents the size of nanoconstructs that make up 10% of the cumulative amount in a drug product). In embodiments the drug product has nanoconstructs that have a particle size distribution that is no greater than about 10 nm, no greater than about 5 nm, and no greater than about 1 nm.

Generally, for PDT the wavelength of the light source needs to be appropriate for exciting the photosensitizer to produce reactive oxygen species. These reactive oxygen species generated through PDT are free radicals or a highly reactive state of oxygen known as singlet oxygen. Typically, in embodiments, the photosensitizer can generate a triplet state of appropriate energy (approximately 0.95 eV) which is the minimum energy required to excite the triplet ground state of molecular oxygen (³O₂) to its excited singlet state (¹O₂). Other cytotoxic species that can be generated include, for example, other ROS, type 1 ROS, hydroxyl radical, peroxides, and superoxide anions.

Typically, there are three mechanisms, which in embodiments can be inter-related, through which PDT mediates the destruction of the targeted tissues, e.g., tumor destructions: direct cytotoxic effects on tumor cells, damage to tumor vasculature, and induction of an inflammatory immune reaction that can lead to the development of systemic immunity.

The incorporation of the photosensitizer in the smaller nanoconstructs, e.g., 8PEGA, provides the ability to have photosensitizers with lower energy states. It is theorized that among other reasons, because the photosensitizer is at or near the surface of the NC it has more tissue oxygen available to form ROS, the smaller size of the NC provides ability for the NC and thus the photosensitizer to generate RO species in closer proximity to the tissue or structure to be affected by the ROS.

In an embodiment, a typical photosensitizer could be, for example, an efficient PDT agent if the quantum yield of singlet oxygen or other reactive oxygen species is high enough (>0.4). That is, at least 40% of excited photosensitizer molecules will create singlet oxygen or reactive oxygen species instead of disbursing energy through fluorescence, phosphorescence or other means. In addition, the longer the life time of the excited photosensitizer's triplet state (>1 ms), the better the interaction with surrounding molecules, resulting in the generation of more cytotoxic species. The embodiments of the present inventions that utilize smaller sized nanoconstructs, and drug products having highly uniform size distributions of the nanoconstructs, provides the ability to have less efficient photosensitizers function as effect PDT, as well as, greatly increase the therapeutic efficacy of existing photosensitizers used in PDT. The incorporation of the photosensitizes in the smaller nanoconstructs, e.g., 8PEGA, provides the ability to have photosensitizes with lower energy states. It is theorized that among other reasons, because the photosensitize is at or near the surface of the NC it has more tissue oxygen available to form ROS, the smaller size of the NC provides ability for the NC and thus the photosensitizer to generate ROS species in closer proximity to the tissue or structure to be affected by the ROS.

It is noted that the mechanism of PDT is distinguished from other light-based and laser therapies such as laser wound healing and rejuvenation or intense pulsed light hair removal, which do not require a photosensitizer, fluorescence, phosphorescence or other means that generate, e.g., create in vivo, or require the generation of an active moiety.

In embodiments, typically, examples of the structures that are targeted by the PDT can be mitochondria, lysosomes or endoplasmic reticulum. The effect on the cell, e.g., cyttid effect, is theorized to occur through, for example, apoptotic cell death mechanism, necrotic paths and both. It is theorized that enzymes needed for apoptosis are destroyed and there will be enough cell damage to cause a necrotic result (plasma membrane damaged). Another of the main causes of tumor destruction is theorized to be through vascular shutdown limiting the supply of oxygen and nutrients to tumor, leading to tissue hypoxia and cell death. A further theorized mechanism is that PDT activates the immune response, which causes infiltration of immune cells such as lymphocytes, leukocytes and macrophages into the targeted tissue. Another of the causes of tumor destruction is through vascular shutdown limiting the supply of oxygen and nutrients to tumor, leading to tissue hypoxia and tumor cell death. In embodiments of the 8PEGA NC in tumor destruction, this is the primary means of tumor cell death.

The embodiments of the present inventions that utilize smaller sized nanoconstructs, drug products having highly uniform size distributions of the nanoconstructs, and combinations of these, provides the ability to use new photosensitizers, older less favored photosensitizers, and photosensitizers that were previously ignored for PDT. These embodiments can use these presently theorized mechanisms of tumor cell death, as well as, other methods or pathways, that will occur, or that may be later discovered, as a result of the present inventions, including the present inventions imaging capabilities and theranostics.

The present invention is not limited to a particular photosensitizing agent. In some embodiments, the agent is methylene blue (MB), chlorin e6 (Ce6), coomassie blue (which in embodiments functions as a PTT (photothermal therapy) agent), gold, or other suitable photosensitizing agents. In some embodiments, the photosensitizing agent is also suitable for imaging (e.g., MB).

Embodiments of the present inventions are not limited to photodynamic therapy. Additional therapeutic agents, that may form a part of the present nanoconstructs and nanoconstruct drug products, may be utilized in embodiment of the present invention. Examples include, but are not limited to, agents that induce apoptosis; sonosensitizers; polynucleotides (e.g., anti-sense, ribozymes, siRNA); polypeptides (e.g., enzymes and antibodies); agents that bind (e.g., oligomerize or complex) with a Bcl-2 family protein such as Bax; alkaloids; alkylating agents; antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2); adoptive immunotherapy agents; hematopoietic growth factors; agents that induce cell differentiation (e.g., all-trans-retinoic acid); gene therapy reagents (e.g., antisense therapy reagents and nucleotides); angiogenesis inhibitors; proteosome inhibitors: NF-KB modulators; anti-CDK compounds; HDAC inhibitors; heavy metals (e.g., barium, gold, or platinum); chemotherapeutic agents (e.g., doxorubicin or cisplatin) and the like. Numerous other examples of toxic compounds are known to those skilled in the art, and use of these compounds by be enabled by the smaller size NC, highly uniform NC size distribution in drug products and combinations of these.

In some embodiments, toxic agents are sonosensitizers. Examples of sonosensitizers include, but are not limited to, porphyrins (e.g., hematoporphyrin, diacetylhematoporphyn-mitomycin-C conjugate, photofrin II, mesoporphyrin, protoporphyrin IX, copper protoporphyrin, tetraphenylporphine tetrasulfonate, ATX-70, ATX-S10, pheophorbide-a, CIA1-phtalocyanine tetrasulfonate, and chlorine PAD-S31), tenoxicam, piroxicam, rose bengal, erythrosine B, merocyanine 540, dimethylformamide, cytosine arabinoside, pyridoxarbazole, 2,2′-azobis(2-amdinopropane), 5,5′-dimethyl-1-pyrroline-X-oxide, e-pyridyl-1-oxide-N-t-butylnitrone, and anti-cancer agents (e.g., nitrogen mustard, cyclophosmadmide, bleomycin, adriamycin, FAD104, amphotericin B, mitomycin C, daunomycin, cisplatin, etopside, diaziquone, dihydroxy(oxbi-guoanido) boron, and 5-fluorouracil) (See e.g., Rosenthal et al., Ultrasonics Sonochemistry 11 (2004) 349; herein incorporated by reference in its entirety).

In some embodiments, toxic agents comprise agents that induce or stimulate apoptosis. Agents that induce apoptosis include, but are not limited to, radiation (e.g., X-rays, gamma rays, UV); tumor-derived growth factor ligands, receptors, and analogs; kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, BEXXAR, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine, dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil), floxuridine (fluorode-oxyuridine), and cytarabine (cytosine arabinoside)); and 3) purine analogs (e.g., mercaptopurine (6-mercaptopurine), thioguanine (6-thioguanine), and pentostatin (2′-deoxycoformycin)).

In still further embodiments, chemotherapeutic agents suitable for use in the compositions and methods of the present invention include, but are not limited to: 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin and carboplatin); 7) anthracenediones (e.g., mitoxantrone); 8) substituted ureas (e.g., hydroxyurea); 9) methylhydrazine derivatives (e.g., procarbazine (N-methylhydrazine)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

In some alternative embodiments, nanoparticles, nanoconstructs and both include additional agents for imaging purposes. In some embodiments, the imaging agent is, for example, selected from magnetic materials (e.g., iron for MRI); proteins that catalyze luminescent reactions (e.g., luciferins such as luciferase for bioluminescent imaging); fluorescent dyes (e.g., rhodamine or fluorescein isothiocyanate for fluorescent imaging); fluorescent proteins (e.g., green fluorescent protein); and radioactive elements (e.g., for autoradiography).

In some embodiments, nanoparticles, nanoconstructs and both comprises nanomaterials to be used as a contrast agent for X-ray/CT, or MRI utilizes photoactive properties, absorbance for X-rays or paramagnetic properties for T1 magnetic resonance imaging. Exemplary contrast agents include, but are not limited to, Gadolinium contrast agents, fluorescent agents (e.g., Alizarin Red S), and contrast agents described in U.S. Pat. Nos. 7,412,279 or 6,540,981, each of which is herein incorporated by reference in its entirety.

Embodiments of the present invention provide activators that activate the toxic agent, leading to local cellular and tissue damage in target cells in a cell specific manner. The present invention is not limited to a particular activator. Any activator that activates the toxic agent finds use in embodiments of the present invention. In general, activators provide a source of energy that results in the toxic agent releasing energy (e.g., in the form of free radicals) that leads to cell death or destruction. Exemplary activators include, but are not limited to, light, heat, radiation, sound, and the like.

In some embodiments, the present invention is illustrated using photodynamic therapy. However, the present invention is not limited to the use of photodynamic therapy. A variety of toxic agents and activating systems finds use in embodiments of the present invention.

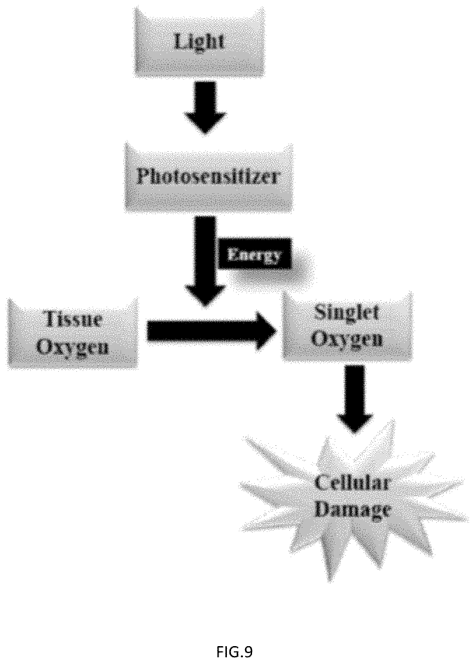

Turning to FIG. 9, in general embodiments of photodynamic therapy (PDT) comprises use of a chemical reaction whereby a photosensitizer is activated by light energy and releases reactive oxygen species. PDT includes two stages. First, the photosensitizing agent is administered and accumulates on or in the tissue by passive or active targeting. Then, the photosensitized tissue is exposed to light at a wavelength that coincides with the absorption spectrum of the photosensitizing agent which, upon illumination, becomes excited. With photodynamically efficient photosensitizers, this leads to an energy transfer to molecular oxygen (available in cells) and to the generation of reactive oxygen species (ROS), mainly singlet oxygen (O₂). The subsequent oxidation of the cell's lipids, amino-acids and proteins induces cellular damage, such as, necrosis and/or apoptosis of the tissue. As ROS, due to an extremely limited lifetime and diffusion length, have a much localized toxicity, their release leads to irreversible but exquisitely restricted cellular damage and tissue necrosis. Thus, the damage induced by PDT is confined to the cells that have been photosensitized, while adjacent non-photosensitized cells remain unaffected.

Embodiments of the present nanoplatforms are conjugate photosensitizers as well as targeting moieties with hydrogels in such a way that targeted, cell-specific PDT is made available for a variety of applications at greatly increase efficacy and controllability. For example, a cell- and spatially-specific cellular death methodology encompassing the synergistic implementation of two agents, both conjugated with a biodegradable nanoparticle: a myocyte-targeting peptide (e.g., CTP), and a photodynamic therapy enabling photosensitizer (e.g., chlorin e6).

In some embodiments, the activator is sound (e.g., sonodynamic therapy). Sonodynamic therapy is the ultrasound dependent enhancement of cytotoxic activities of certain compounds (sonosensitizers). Ultrasound is a mechanical wave with periodic vibrations of particles in a continuous, elastic medium at frequencies equal to or greater than 20 kHz. In liquids, its velocity of about 1000-1600 m/s translates into the wavelength range from micrometers to centimeters. Consequently, the acoustic field cannot couple directly with the energy levels of molecules, including the biological milieu at the molecular level. Therefore, this radiation is not only perceived as safe, but has a very good tissue penetrating ability without major attenuation of its energy. In some embodiments, sound is generated outside of the body and targeted through tissue to the desired treatment region.

Sonodynamic therapy is based on the synergistic effect of ultrasound and a chemical compound referred to as “sonosensitizer”. The effect can be localized by focusing the ultrasound on a defined region (e.g., regions of target tissue). In some embodiments, ultrasound is delivered transdermally to a specific region of target tissue.

In some embodiments, activators are pharmaceutical agents that activate therapeutic agents (e.g., chemotherapeutic agents). For example, in some embodiments, verapamil is used to active or improve efficacy of chemotherapeutic agents (e.g., doxorubicin).

Embodiments of the present invention provide compositions, kits, and systems comprising the nanoconstructs and nanoconstruct drug products described herein. In some embodiments, systems comprise nanoparticles, nanoconstructs and both and instruments or apparatuses for delivering the activator (e.g., laser, ultrasound apparatus, radiation delivery apparatus and the like). In some embodiments, systems further comprise instruments for imaging nanoparticles, nanoconstructs and both in targeted tissue and computer systems to control delivery of activators, imaging, data analysis, and data display.

Cell-specific death is then induced upon local delivery of activator (e.g., laser light or sound) delivery (e.g., via the toxic agent embedded or on the surface of the nanoparticle), followed by local release of ROS. In embodiments, specifically targeted cell types are killed only in the areas where activator is delivery, while the number of untargeted cells stays constant after delivery of the activator.

The present invention is not limited to a particular method of delivery of activator. In some embodiments, activator is delivered directly to the areas of the target tissue in need of therapy via surgery, e.g., endoscopic or open. In some embodiments, activators are targeted and controlled using automated systems (e.g., computer controlled).

In some embodiments, activators are delivered locally to the targeted areas in need of treatment using a catheter or other intravenous or intraarterial delivery or transdermally (e.g., via ultrasound). Such methods avoid the need for open surgery.

In some embodiments, therapy is sonodynamic therapy. Sonodynamic therapy has the advantage of transdermal delivery, thus allowing the entire procedure to be conducted without invasive means. The toxic agent (e.g., sonosensitizer) is delivered (e.g., intravenously) and then targeted areas of tissue are treated with ultrasound.

In some embodiments, therapy is photodynamic therapy. Photodynamic therapy has the ability to bring spatial specificity, as only the areas illuminated are receiving therapy, while other regions remain untreated.

In some embodiments, therapies target and ablate or kill myocytes. However, the present invention is not limited to the targeting of myocytes. An advantage of implementing therapeutic nanoplatforms is the high versatility of these carriers to be conjugated to various optional targeting agents, for distinct target tissue applications. In fact, any other targeting moieties (e.g., antibodies, peptides, etc.), functional dyes or bioactive agents can be readily implemented with these nanoplatforms.

In some embodiments, therapeutic uses described herein are used in conjunction with existing therapies or as a replacement for existing therapies. In some embodiments, nanoparticle-based therapeutics are used as a follow-up to failed or incomplete therapy (e.g., non-nanoparticle therapies).

In some embodiments, nanoparticles, nanoconstructs and both are utilized in imaging (e.g., in vivo imaging) applications. In some embodiments, a photosensitive agent (e.g., chlorin e6) or particle that is also fluorescent or otherwise imageable is utilized. As described herein a significant embodiment of the present inventions are the nanoparticles, nanoconstructs, and both that themselves, with the addition of other agents or materials, function as an MRI imaging agent. In other embodiments, nanoparticles, nanoconstructs and both further comprise separate imaging agents. For example, as described herein, in some embodiments, nanoparticles, nanoconstructs and both comprise contrast agent for imaging (e.g., X-Ray, computer tomography (CT) imaging, PET imaging, ultrasound, photo-acoustic imaging, or MRI imaging). For example, in some embodiments, ¹⁵⁷Gd, gold, iodine, iron-oxide, or other suitable agent for use in imaging coat nanoparticles, nanoconstructs and both.

In some embodiments, nanoparticles, nanoconstructs, and both are used to detect biological targets in vivo or in vitro by bioluminescent imaging. In some embodiments, nanoparticles, nanoconstructs and both comprise an enzyme that catalyzes a bioluminescent reaction. Enzymes that catalyze bioluminescent reactions include, but are not limited to, the following luciferases: bacterial luciferase (U.S. Pat. No. 4,548,994), Photinus pyralis luciferase (U.S. Pat. Nos. 5,670,356 and 5,674,713), Renilla reniformus luciferase, Pyrophorus plagiophthalamus luciferase, Luciola cruciata luciferase (Masuda et al., Gene 77:265-70 [1989]), Luciola lateralis luciferase (Tatsumi et al., Biochim. Biophys Acta 1131:161-65 [1992]), and Latia neritoides luciferase. The foregoing publications and patents are specifically incorporated herein by reference.

In some embodiments, the imaging is performed in situ. Nanoparticles, nanoconstructs and both containing the bioluminescent enzyme are provided to the animal intravenously and allowed time so that the molecular recognition element binds to its biological target. In some embodiments, a substrate (e.g., bacterial or insect luciferin) for the bioluminescent enzyme is then provided (e.g., via intravenous, intraperitoneal, intravesical, or intracerebrovascular delivery) to the animal. In some embodiments, production of bioluminescence by the action of the enzyme on the substrate is then detected by a bioluminescence detection system. In some embodiments, the bioluminescence detection system comprises a Hamamatsu intensified CCD (ICCD, model C2400-32). In other embodiments, the bioluminescence detection system further comprises other devices for intensifying weak signals (e.g., microchannel plate intensifiers and devices for Peltier or liquid nitrogen cooling of the detector and/or intensifier). In some embodiments, a grey scale image of the animal is obtained by opening the door of dark chamber in which the animal is placed. The door is then shut and the gain on the intensifier adjusted to maximum to detect the bioluminescent signal. The signal is then overlaid with the greyscale image in pseudocolor.

In other embodiments, nanoparticles, nanoconstructs and both are used to detect biological targets by magnetic resonance imaging (MRI). In some embodiments, the biological target imaged is in situ. Nanoparticles, nanoconstructs and both comprising the magnetic material are provided to the animal (e.g., intravenously) and time allowed so that the molecular recognition element binds to its biological target. In some embodiments, the biological target is then imaged with a magnetic resonance system (e.g., a 7-Tesla Magnetic Resonance System). In some embodiments, T₁-weighted or T₂-weighted images are obtained.

In some embodiments, diagnostic and imaging applications are performed in combination with therapeutic applications. For example, in some embodiments, imaging agents are utilized to visualize target tissue before and after photodynamic therapy to monitor cell death. For example, in some embodiments, imaging allows a clinician to see where nanoparticles, nanoconstructs and both are bound (e.g., before, during or after activation). In some embodiments, imaging is used to visualize target tissue after treatment to determine the extent or localization of cell killing. Thus, the imaging allows real time monitoring of the progress of the dynamic therapy.

In some embodiments, imaging methods are utilized to determine a treatment course of activation. For example, in some embodiments, imaging is used after treatment to determine if additional treatment is needed in the form of, for example, additional activator in the same or different regions or delivery of additional nanoparticles, nanoconstructs and both.

In some embodiments, nanoparticles, nanoconstructs and both are used in research (e.g., imaging in animal models, structural studies, DNA-protein binding interactions, protein capture, etc.), during surgery, or drug screening applications.

Various annalistic and monitoring techniques and equipment can be used to evaluate the present NCs, methods of makings these NCs, and methods of using these NCs. For example, a Shimadzu UV-1601 UV/Visible Spectrophotometer can be used for recording and adjusting the optical density (OD) of NPs. Fluorescence spectra can be taken using a Fluoromax-3.

Starting materials, reagents and components used to make the present NCs can come from available commercial sources, and preferably are FDA approved materials for use in medical products. For example, the following are sources of the materials that can be used for the embodiments in the Examples. Chlorin e6 (Ce6) and 1-Ethyl-3-(3-dimethylaminopropyl)carbodiimide (EDC) are sourced from Frontier Scientific. 8PEGA (40 kDa) and Bi-PEG (Maleimide-PEG-Succinimydal Ester, 2kDA) is sourced from Creative PEG Works. F3-Cys peptide (KDEPQRRSARLSAKPAPPKPEPKPKKAPAKKC) is sourced from SynBioSci. 190 proof natured ethanol from Decon Labs. 10 kDa and 300 kDa filters for Amicon Cells and 10 kDa centrifugal filters are sourced from Amicon. DMEM [(+) glutamine, sugar, sodium pyruvate), penicillin streptomycin, and fetal bovine serum are sourced from Life Technologies. All other chemicals sourced from Sigma Aldrich. Acrylamide, 3-(Acryloyloxy)-2-hydroxypropyl methacrylate (AHM), aminopropyl methylacrylamide hydrogen chloride salt (APMA), N-hydroxy succinimide (NHS), N,N′-Dicyclohexylcarbodiimide (DCC), Brij L4, dioctyl sulfosuccinate sodium salt (AOT), dimethyl sulfoxide (DMSO), dimethyl formamide (DMF), ammonium persulfate (APS), tetramethyl ethylene diamine (TEMED), phosphate buffer saline (0.01 M, PBS), hexanes, cysteine, anthracene dipropionic acid (ADPA), calcein AM, propidium iodide (PI).

In an embodiment, the NMR is operated and analyzed to provide sufficient water and fat suppression to provide a clean PEG image. For example, a diffusion-based method can be employed where 95% of tissue water is eliminated. The residual water can be removed by other water suppression methods know to those of skill in the art. For example, to have additional water suppression, spectroscopic imaging where each voxel consist of an NMR spectrum with water, PEG, and fat can be used. This method of suppression, also has applicability in tissue distribution studies.

In an embodiment the MRI of the 8PEGA is directly picking up on the signal of the ethoxy protons and measuring it. This is different from metals such as gadolinium, which changes the relaxation time of the water protons it interacts with, thereby creating a contrast that is visible during scanning. Thus, an embodiment of the present invention provides for images based upon the direct measurement of protons in an NP, and does not rely upon, and does not use, changes in the relaxation time of water protons.

Embodiments of the NC, and in particular the PEG based NC, can be various different sizes and weights, and would include, for example, monomeric (e.g., single, large macromolecules that are not constructed of multiple PEGs), multi-armed PEGs (e.g., 2, 4, 6, 8, 10 or more arms), and combinations and variations of these. The NCs can have total MW of <1 MDa, from about 10 kDA to about 1,000 kDa, from about 20 kDa to about 500 kDa, from about 30 kDa to about 200 kDa, from about 40 kDa to about 750 kDa, from about 35 kDa to about 75 kDa, from about 50 kDa to about 800 kDa, and all values within these ranges, and larger and smaller amounts.

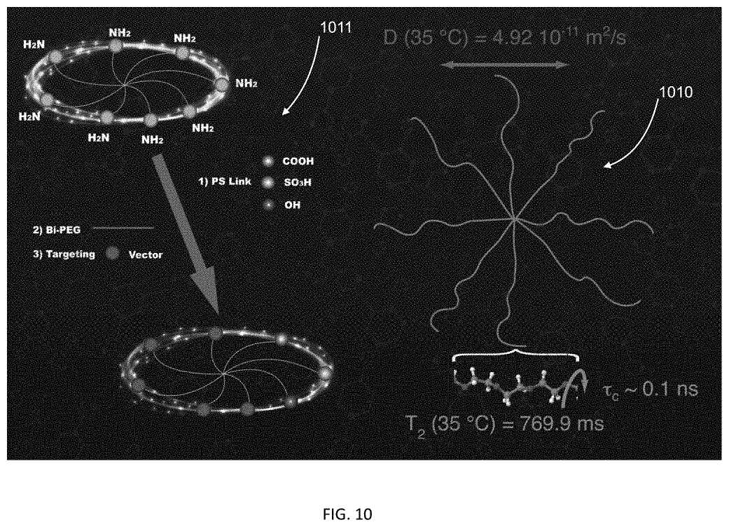

Turning to FIG. 10, there is provided a chart illustrating a an embodiment of an 8-arm-PEG-amine (8PEGA) 1010, which is a universally applicable ultra-small nanoplatform for theranostics: e.g., photodynamic therapy (PDT, “thera”), and as a molecular MR agent (“nostic”). The left of the image 1011 illustrates the flexibility with which a photosensitizer (PS) can be attached via different functional groups, and then a targeting agent (e.g. peptides) attached through simple “click” chemistry reactions with bi-functional PEG, like the maleimide-thiol reaction, for cell-specific targeting methodology. The right shows how 8PEGA 1010 contains various physical properties ideal for MRI imaging. These properties include a long T₂ life-time and a slow diffusion constant due to its high MW of 40 kDa, ideal properties for diffusion weighted MRI.

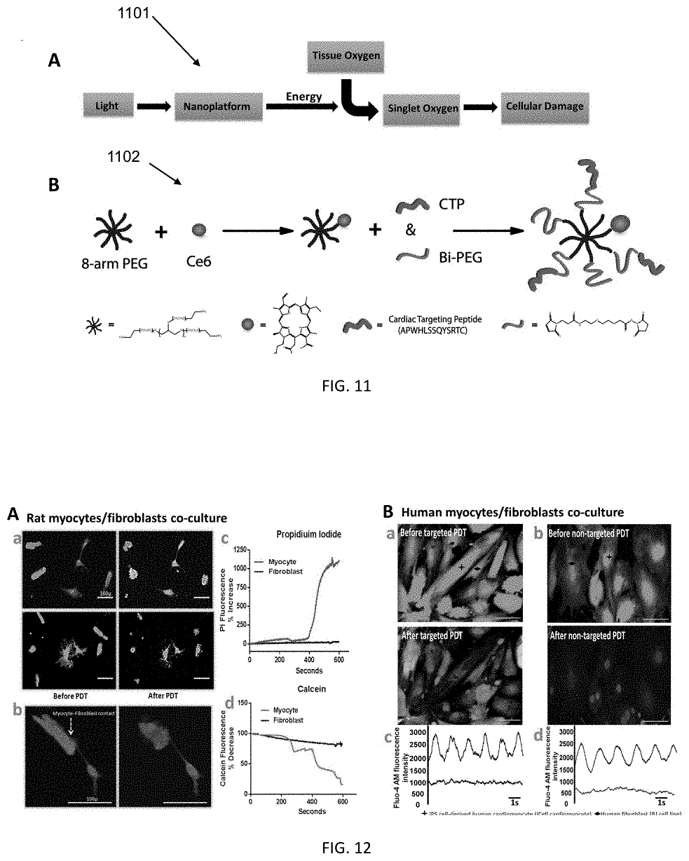

Turning to FIG. 11, there is shown a generic flow chart of assembling 8PEGA with Ce6 (PS) and CTP (TA). This can be generically applied to using any suitable TA and PS. The top chart 1101 is a generic route description of how PDT functions: A nanoplatform receives light, which it absorbs and transfers to local oxygen to create ROS. This ROS then damages the cells. The bottom chart 1102 is a schematic reaction path for making a targeted NC.

Turning to FIG. 12 there is shown a slide of the performance of the CTP-8PEGA-Ce6 NP in vitro on rat and human cell cultures; the cultures include both fibroblasts and cardio myocytes. It can be seen in the rat case that myocytes only are killed, even when in direct contact with a fibroblast. A dead cell will see a decrease in calcein AM fluorescence and increase from propidium iodide. The human cell test shows that this same selectivity can be achieved in a human without the need for changes to the platform (targeted and non-targeted tests were carried out, showing targeting was both preferred and effective).

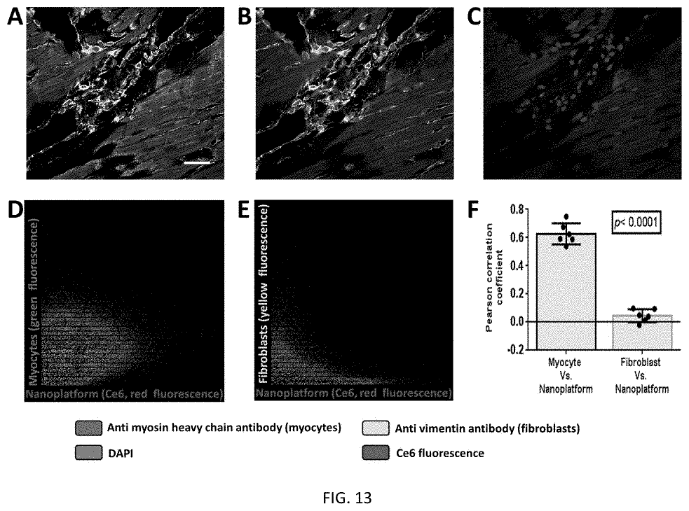

Turning to FIG. 13 there is shown tissue from rat hearts that were isolated after a NP injection (1 hr post injection). DAPI stains the nucleus of all cells. It can be seen that the fluorescence of Ce6 is localized with extreme selectivity to the myocytes, indicating that the in vitro selectivity results translate directly to in vivo.

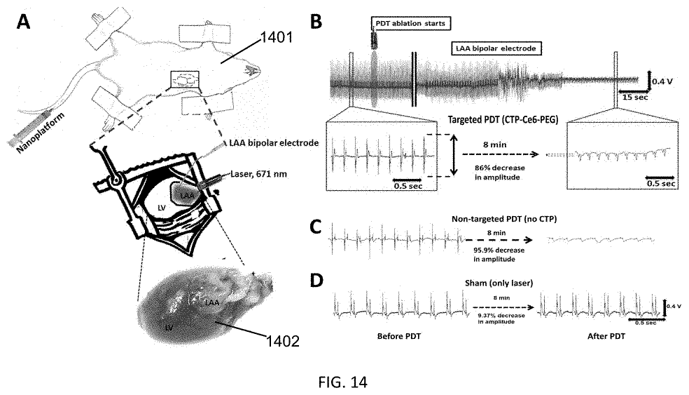

Turning to FIG. 14, there is shown a demonstration of the surgical setup subject 1401, heart 1402, and the resulting electrograms from PDT (either targeted, non-targeted, or sham). A decrease in the electrogram amplitudes indicates signal blockage; residual signals were attributed to far field activity. Both targeted and non-targeted had an effect, while laser alone did not. This indicates that PDT in general can cause a change.

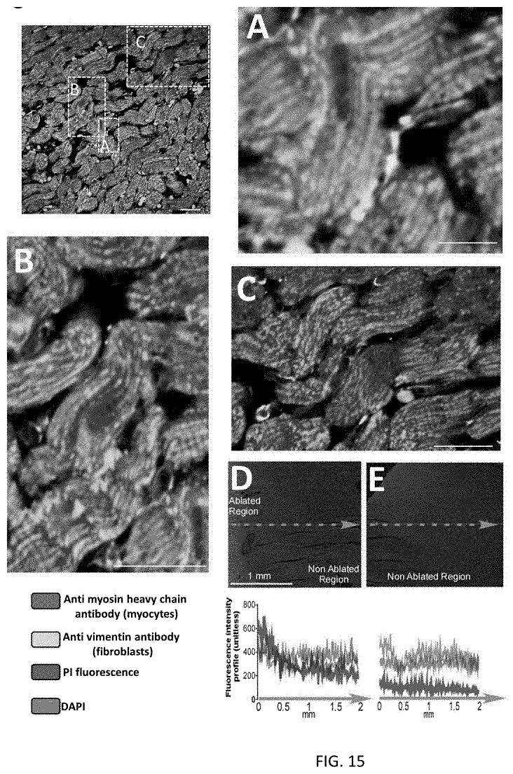

Turning to FIG. 15, there is shown that PI (a nuclear stain for dead cells) is only present in myocytes (3 different magnified areas). D and E show that PDT only occurred in the illuminated region by showing the PI fluorescence decrease as the examined area is shifted away from the treatment area.

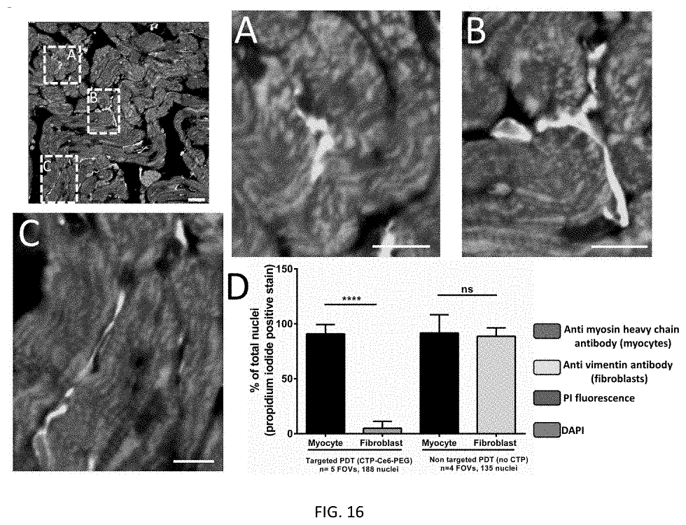

Turning to FIG. 16, there is shown similar tissue sampling test as FIG. 15, but using non-targeted PDT. It is seen in the bar the pictures (and quantified in the graph) that untargeted Ce6 PDT kills everything, compared to targeted which only kills myocytes.

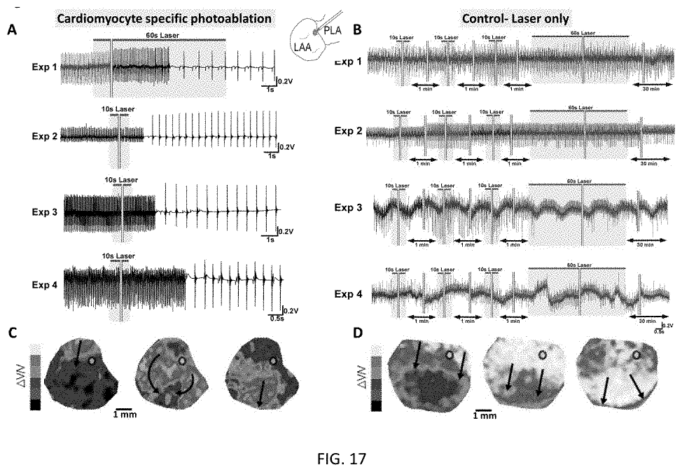

Turning to FIG. 17, there is shown cardiograms of mice that receive either targeted PDT or laser only. Only with the targeted PDT is there a difference in the heart beat returning to normal.

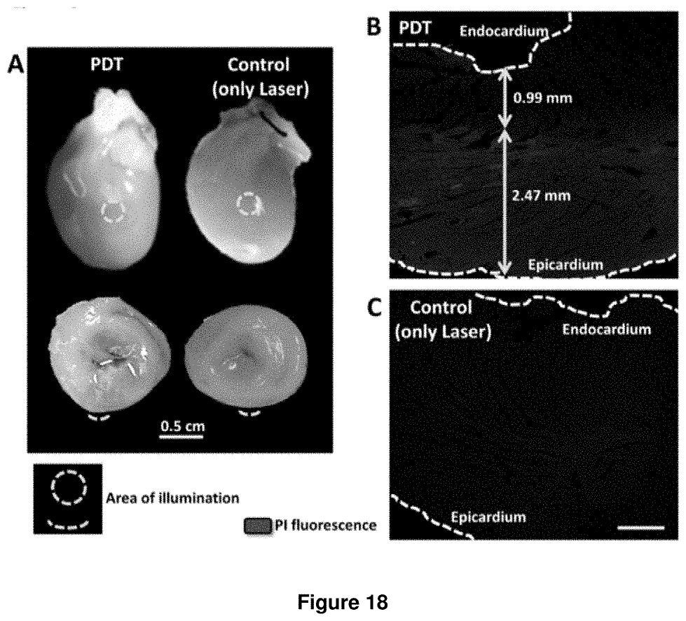

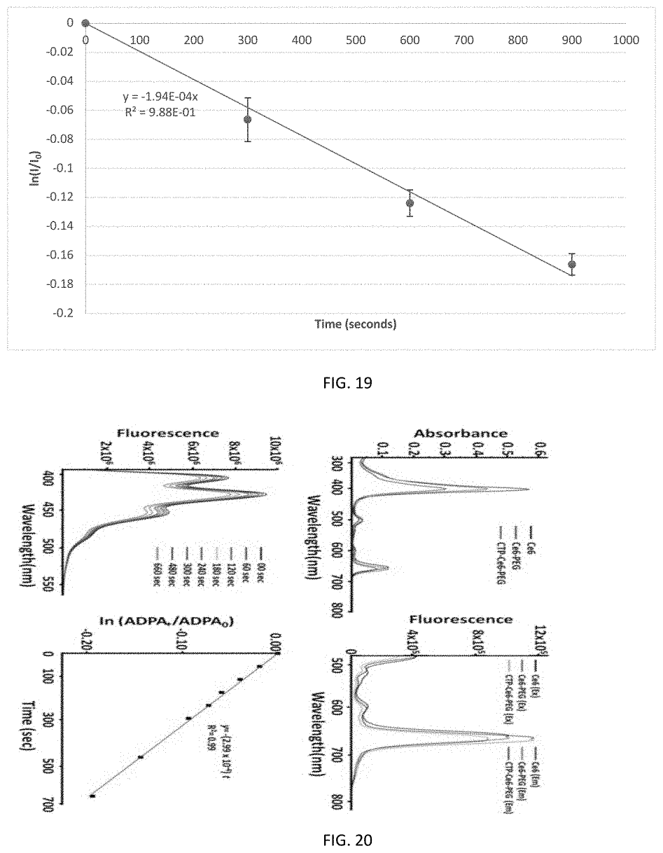

Turning to FIG. 18, there is shown how deep the PDT penetrated the tissue (tracked by PI fluorescence through the tissue). Also there is shown that the laser alone had no effect.

In an embodiment there is provided a new targetable nanoconstruct (NC) capable of simultaneously serving as a therapeutic platform for photodynamic therapy (PDT) as well as an MR molecular imaging agent, free of heavy metal atoms. In an embodiment there is provided NC based PDT for cancer. In an embodiment there is provided a F3-cys targeting agent NC. In an embodiment, the 8PEGA-Ce6 NCs have a superior reactive oxygen species (ROS) production compared to traditional Ce6 encapsulated Polyacrylamide (PAAm) NCs. This embodiment of an NC is also cyto-compatible and offers chemical flexibility for the attachment of a choice of targeting peptides. There is also provided a label-free 8PEGA NC that can be directly and selectively imaged by MRI, using standard spin-echo imaging sequences with large diffusion magnetic field gradients to suppress the water signal. Notably, due to its ultra-small size this NC can have improved in vivo penetration and bio-elimination.

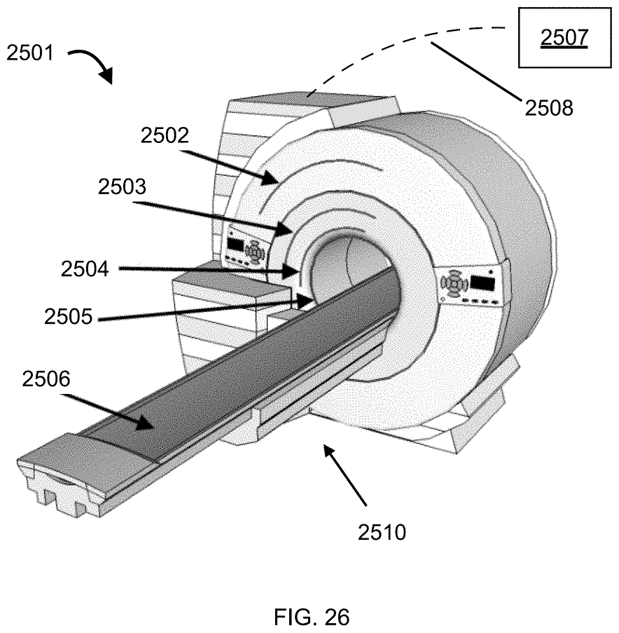

Turning to FIG. 26, there is shown a schematic diagram of an embodiment of an MRI system 2501. The system 2501 has a magnet 2502, gradient coils 2503, radio frequency coils 2504, a bore 2505 and a table 2506. It being understood that this figure is a schematic representation, that other components and other configurations and types of MRI systems may be employed. The system 2501, in an embodiment, is configured to generate three magnetic fields. The first field is a strong static magnetic field to create energy level differences in nuclei with spin angular momentum and gives rise to bulk nuclear magnetization. The second field is a radio frequency field and is used to tip the created nuclear magnetization so that it can be detected by RF coils 2504. The third field is a set of magnetic field gradients is used to spatially encode the signal to create a map of nuclear magnetization. Thus, in embodiments the magnetic fields are configured to generate an image of non-water protons present in an additive placed in a subject to be imaged; wherein the magnetic field gradients can be pulsed in a specific manner to sensitize the nuclei to motion due to flow or diffusion.

In an embodiment an imaging agent made up of the present nanoconstructs, e.g., PEG based nanoparticles, is administered to a patient. The patient with the administered imaging agent is then placed on table 2506, the table is moved into the bore 2505, and the system 2501 performs a scan, obtain MR images, of for example the types disclosed in this specification. The nanoparticles are directly imaged providing a detailed image, and data, regarding their position in the patient. From this information a therapy can be designed, refined and implemented. For example, if the nanoconstructs were targeted PDT nanoconstructs a surgical, surgical an PDT, or PDT alone, therapy could be developed and them implemented to remove the targeted tissue.

The MRI system 2501 has a control system 2507, which includes operator input and other control features, as well as operating instruction, such as computer code. The control system 2597 is in control communication with the device 2510, as shown by dashed line 2508. By control communications it is meant that data, information and control commands as well as other instructions are communicated between the control system 2507 and the device 2510. The control system 2507 may be separate from the device 2510, or it may be a part of the device 2510, e.g., within the structure of the device 2510. In an embodiment, instructions to operate the MRI to provide the operating parameters for imaging PEG based nanoparticles are provided to the system 2501. This can be way of a software upgrade, for example. In this manner existing MRI systems can be readily upgrade to take full advantage of the benefits of the present PEG based nanoparticle image agents.

### EXAMPLES

The following examples are provided to illustrate various embodiments of systems, therapies, processes, compositions, applications and materials of the present inventions. These examples are for illustrative purposes, may be prophetic, and should not be viewed as, and do not otherwise limit the scope of the present inventions. The percentages used in the examples, unless expressly provided otherwise, are weight percents of the total, e.g., formulation, composition, mixture, product, or structure, unless specifically stated otherwise.

### Example 1

Embodiments of Ce6-8PEGA NCs are ultra-small and possess superior ROS production, compared to encapsulated PAAm-Ce6 NPs. The successful exchange of the targeting peptides, from CTP to F3-cys, demonstrates this NC's chemical flexibility in changing targets. The F3-8PEGA-Ce6 NCs has good biocompatibility in vitro and in vivo.

### Example 2

Embodiments of 8PEGA NPs and NCs are molecular imaging agent in MRI. These imaging agents can further be coupled with techniques to suppress water and fat signals, providing even enhanced imaging capabilities.

### Example 3

An F3-8PEGA-Ce6 presents an attractive universal NC for theranostics (imaging and PDT), from heart arrhythmia to cancer, and possibly to other pathologies. Benefits, include among others, rapid renal clearance and of accumulation in early stage tumors, even before angiogenesis, coupled with the MRI results.

### Example 4

8PEGA-Ce6 Conjugate: Ce6 is conjugated to 8PEGA via DCC/NHS coupling in DMF. Briefly, 448 uL of Ce6 solution (20 mg/mL, DMF) is activated with 154.8 uL DCC and 172.8 uL NHS under stirring (20 mg/mL, DMF) for 30 minutes. 500 mg 8PEGA is solvated in DMF at a concentration of 50 mg/mL using sonication. Upon solvation, the Ce6 solution is added to the 8PEGA solution and allowed to stir overnight. The following day, unconjugated Ce6 is removed using 50% ethanol/PBS mixture in an Amicon Cell filtration system using a 10 kDa membrane. After purification, the solvent is exchanged with Millipore ultrapure water, the materials filtered using a 0.45 um syringe filter, and freeze dried for storage.

### Example 5

F3-8PEGA-Ce6 Conjugate: 8PEGA-Ce6 was modified with F3 via the same methods reported by our lab over the years. After modification with F3-cys, the UV/VIS was then taken to ensure that the Ce6 had not aggregated in the process. Briefly, 20 mg of Bi-PEG is added to 1 mL of 8PEGA-Ce6 (20 mg/mL, PBS) and stirred for 30 minutes. The solution is then washed 4×15 minutes in PBS using a 10 kDa centrifugal filter. The resulting solution is concentrated to 20 mg/mL (by original mass), 22 mg of F3-cys is added (220 uL, 11 mg/110 uL DMSO), and left to stir over night. The next day, excess of cysteine is added and stirred for 2 hours to cap any unreacted maleimide groups. The solution is then filtered again using a 10 kDa centrifugal filter and millipore ultrapure water, and freeze dried for storage.

### Example 6

Ce6 Encapsulated Polyacrylamide Nanoparticles (PAAm NPs): Ce6 is encapsulated in PAA NPs through a slightly modified previously reported method. Briefly, 5 mg of Ce6 is added to 930 uL of PBS and 100 uL DMSO with 28 mg APMA, 19 mg NHS, and 16 mg EDC. The solution is stirred at 37° C. for 2 hours. Acrylamide and AHM are then added to the solution (368 mg and 52.6 uL, respectively) and sonicated to create a uniform solution. This solution is added to a 100 mL round bottom flask containing 31 mL hexanes, 2.2 mL Brij L4, and 1.07 g AOT under stirring. The stirring is adjusted to where the generated vortex is just barely touching the stir bar (˜500 RPM). The contents of the flask are then purged with nitrogen for 15 minutes. Nitrogen flow is removed from contact with the flask contents and maintained inside the flask. 15 mg of APS in 100 uL of water is added dropwise to initiate polymerization and 100 uL of TEMED added dropwise to catalyze the process. Polymerization is allowed to proceed for 2 hours. Hexanes are then removed via rotary evaporation. The resulting contents are re-dispersed in ethanol and cleaned using 10×150 mL ethanol and 5×150 mL Millipore ultrapure water in an Amicon Cell using a 300 kDa filter. The purified materials are syringe filtered using a 0.45 um filter and freeze dried for storage.

### Example 7

A drug product contains F3-8PEGA-Ce6 NC. The NC have an average particle size of about 10 nm-15 nm, and are uniform, having a very narrow particle size distribution, less than about 5% average distance.

### Example 8

A drug product contains F3-8PEGA-Ce6 NC. The NC have a particle size distribution of D10=10 nm, D50=12 nm, D90=15 nm.

### Example 9

A drug product contains F3-8PEGA-Ce6 NC. The NC have an average particle size within the range of about 10 nm-15 nm, and are uniform, having a very narrow particle size distribution, less than about 10% average distance.

### Example 9A

A drug product contains F3-8PEGA-Ce6 NC. The NC have an average particle size within the range of about 8 nm-18 nm, and are uniform, having a very narrow particle size distribution, less than about 10% average distance.

### Example 9B

A drug product contains F3-8PEGA-Ce6 NC. The NC have an average particle size within the range of about 8 nm-10 nm, and are uniform, having a very narrow particle size distribution, less than about 10% average distance.

### Example 9C

A drug product contains F3-8PEGA-Ce6 NC. The NC have an average particle size within the range of about 10 nm-12 nm, and are uniform, having a very narrow particle size distribution, less than about 10% average distance.

### Example 9D

A drug product contains F3-8PEGA-Ce6 NC. The NC have an average particle size within the range of about 11 nm-14 nm, and are uniform, having a very narrow particle size distribution, less than about 10% average distance.

### Example 9E

A drug product contains F3-8PEGA-Ce6 NC. The NC have an average particle size within the range of about 15 nm-18 nm, and are uniform, having a very narrow particle size distribution, less than about 10% average distance.

### Example 10

A drug product contains F3-8PEGA-Ce6 NC. The NC have a particle size distribution of D10=about 8 nm, D50=about 10 nm, D90=about 12 nm.

### Example 11

A drug product contains F3-8PEGA-Ce6 NC. The NC have a particle size distribution of D10=about 5 to 12 nm, D50=about 8 to 15 nm, D90=about 12 to 22 nm.

### Example 11A

A drug product contains F3-8PEGA-Ce6 NC. The NC have a particle size distribution of D10=about 6 to 10 nm, D50=about 8 to 15 nm, D90=about 12 to 20 nm.

### Example 11B

A drug product contains F3-8PEGA-Ce6 NC. The NC have a particle size distribution of D10=about 10 to 12 nm, D50=about 11 to 13 nm, D90=about 10 to 15 nm.

### Example 12

The drug products of Examples 7 to 12 are used as an imaging agents, contrast agents, or both for NMR or MRI.

### Example 13

Other NCs, imaging agent, or therapeutic agents are included in the drug products of Examples 7 to 12.

### Example 14

The drug products of Examples 7 to 12 can be used with other drug products, such as other NCs, imaging agents, or therapeutic agents.

### Example 15

A drug product contains a TA(targeting agent)-8PEGA-AA(active agent) NC. The NC have an average particle size of about 10 nm, and are uniform, having a very narrow particle size distribution, less than about 5% average distance.

### Example 16

A drug product contains a TA-8PEGA-AA NC. The NC have a particle size distribution of D10=10 nm, D50=12 nm, D90=15 nm.

### Example 17

A drug product contains TA-8PEGA-AA NC. The NC have an average particle size within the range of about 10 nm-15 nm, and are uniform, having a very narrow particle size distribution, less than about 10% average distance.

### Example 18

A drug product contains TA-8PEGA-AA NC. The NC have a particle size distribution of D10=about 8 nm, D50=about 10 nm, D90=about 12 nm.

### Example 19

A drug product contains TA-8PEGA-AA NC. The NC have a particle size distribution of D10=about 6 to 10 nm, D50=about 8 to 15 nm, D90=about 12 to 20 nm.

### Example 20

The drug products of Examples 15 to 19 are used an imaging agent, contrast agent, or both for NMR or MRI.

### Example 21

Other NCs, imaging agent, or therapeutic agents are included in the drug products of Examples 15 to 19.

### Example 22

The drug products of Examples 15 to 19 can be used with other drug products, such as other NCs, imaging agents, or therapeutic agents.

### Example 23

The drug products of Examples 15 to 22, in which the TA is one or more of the following, for example: any peptide that is cysteine terminated and contains no additional free thiol groups can be attached to 8PEG and used, e.g., RGD, cRGD, iRGD, F3, and CTP. Further examples are contained in phage libraries that are publicly available. In general, any cell for which there is a small molecule or peptide that will selectively accumulate in it, can attach it to, or be a part of the NC, and thus function as a TA, including as a TA for 8PEGA.

### Example 24

The drug products of Examples 15 to 22, in which the AA is one or more of the following: a photosensitizer, a sonosensitizer, a photoacoustic agent, and others disclosed in this specification, known to those of skill in the art, or later developed.

### Example 25

An NC having particle size of about 8-15 nm, and having an AA from the group of Example 24.

### Example 26

An NC having particle size of about 8-15 nm, and having an TA from the group of Example 23.

### Example 27

An NC having particle size of about 10-20 nm, and having an AA from the group of Example 24.

### Example 28

An NC having particle size of about 10-20 nm, and having an TA from the group of Example 23.

### Example 29

A drug product having NCs from Examples 25, 26, 27 and 28, with particle size distribution has the D10 and D90 values within 5 nm of the D50 value.

### Example 30

A drug product having NCs from Examples 25, 26, 27 and 28, with particle size distribution has the D10 and D90 values within 10 nm of the D50 value.

### Example 31

A drug product having NCs from Examples 25, 26, 27 and 28, with particle size distribution has the D10 and D90 values within 2 nm of the D50 value.

### Example 32

MRI calibration to enhance quantitative imaging of embodiments of the present imaging agents. Images and spectra produced by selective 8PEGA NMR imaging and spectroscopy sequences and protocols will have signal intensities that are proportional to the local concentration of the PEG nanostructs. If a quantitative measure of the absolute concentration of PEG nanostructs in mg/ml or in molarity is desired, the MRI systems can be calibrated by performing the selective PEG pulse sequences in a particular MRI scanner with a phantom of known PEG concentration or by imaging of the patient and phantom of known PEG concentration at the same time. Moreover, a concentration calibration curve can be constructed as in FIGS. 6 and 7 showing the signal intensity generated by the PEG selective MRI is proportional to the concentration of 8PEGA.

### Example 32A

An MRI is configured to image the protons in a PEG based imaging agent. MRI is an imaging tool usually applied to providing diagnostic medical information to physicians to aid in patient care management. MRI uses three magnetic fields. First, a strong static magnetic field to creates energy level differences in nuclei with spin angular momentum and gives rise to bulk nuclear magnetization. Second, a radio frequency (RF) field is used to tip the created nuclear magnetization so that it can be detected by RF coils. And finally, a set of magnetic field gradients is used to spatially encode the signal to create a map of nuclear magnetization. In addition, the magnetic field gradients can be pulsed in a specific manner to sensitize the nuclei to motion due to flow or diffusion. MRI pulse sequences consist of a series of RF and gradient pulses to generate MR images that are sensitized to T₁, T₂, diffusion, and other parameters.

### Example 32B

One embodiment of an MRI pulse sequence for generation of 8PEGA specific images is seen in FIGS. 6 and 7. In this particular case, a diffusion weighted spin-echo imaging sequence was used with a repetition time TR=500 ms, an echo time TE=200 ms, a pair of diffusion encoding gradients with amplitude G_{diff}=126 mT/m, duration δ=7.1 ms, and separation ∇=180 ms to generate a diffusion b value of 10¹⁰ s/m². The magnetic field gradients attenuate the MR signal intensity by S(b)=exp(−bD) where

$$b = {( γ {δG}_{diff} )}^{2} ( Δ - \frac{δ}{3} ) .$$

The diffusion constant, D, of water is 2.2 10⁻⁹ m²s⁻¹ at 25° C. and 8PEGA is 3.5 10⁻¹¹ m²s⁻¹ two orders slower than water. With these specific parameters, the pure water signal will be reduced by exp(−bD)=2.7 10⁻¹⁰, effectively putting the water signal into the noise floor. Conversely, the 60% of the PEG8A signal remains at b=10¹⁰ s/m². A background image of both PEG8A and water was obtained with identical parameters except that the gradient strength was reduced to 12.5 mT/m, creating a b value of 10⁸ s/m². With these parameters, water is attenuated to 80% of its initial value and PEG8A to 99%, but water proton concentration is significantly higher and dominates the MRI signal.

In vivo, water molecule diffusion is restricted by the cell walls and it is estimated that approximately 5% of the water signal will remain at b=10¹⁰ s/m². Water proton T₂ times are typically <80 ms whereas 8PEGA T₂ times are >500 ms. Therefore once can achieve additional water suppression by imaging at long TE times. The signal attenuation will go as S(b, TE)=exp(−TE/T₂)exp(−bD) so additional water attenuation can be achieved by imaging at long TE times. In addition, conventional water suppression methods based on chemical shift or water proton relaxation times can be used at the same time as gradient suppression methods and to provide even more water signal attenuation.

In clinical MRI systems, the amplitude of the magnetic field gradient is limited to values of approximately 40 mT/m. To generate b values of 10¹⁰ s/m² the values of δ and ∇ will need to be adjusted. For instance, with a gradient amplitude 40 mT/m, δ=60 ms, and ∇=200 ms results in a b value of 7×10¹⁰ s/m², sufficient for selective imaging of 8PEGA.

In general, any MRI pulse sequence that provides high b values and TE times >100 ms will provide sufficient attenuation of water and fat signals.

### Example 33

Photo-acoustic imaging—Photoacoustic imaging (PAI) provides greater depth limits than from other optical imaging systems while also increasing resolution. PAI uses the acoustic waves generated in response to the absorption of pulsed laser light, and provides noninvasive images of absorbed optical energy density at depths of several centimeters with a resolution of for example about 100 μm, and potentially greater.

An 8PEGA photo-acoustic imaging NC (PAI-NC) has as an active agent. The PAI-NC has a particle size of from about 10 nm to 20 nm. The PAI-NC can also have a targeting agent, include one of the targeting agents from Example 23.

The active agent, e.g., imaging agent, contrast agent, for the PAI-NC can be, for example, small-molecule dyes, gold, carbon, liposome encapsulations, heptamethine cyanine dyes (e.g., indocyanine green), azo dyes (e.g., methylene blue), and naphthalolcyanine dyes.

Generally, in selecting an active agent, if there is a hydrophilic R group on the compound structure, it should be possible to tag to PEG (most common groups are sulfonic acid and carboxylate). Cyanine dyes usually contain sulfonic acid groups (SO₃—) which can be derivatized to a chlorinated intermediate that would be highly reactive to the amines on PEG. ICG (indocynine green, available under the tradename CARDIOGREEN) is an example of an FDA approval, spectral absorption (780 nm), agent for use in the 8PEGA PAI-NC. Others that may be used in the PAI-NC include, for example, Coomassie brilliant blue, (abs max=595-610 nm), alexa fluor 750, IR780, and IRDye 800, Ce6, methylene blue, and SiNc.

### Example 34

A system and method for high resolution and precise theranostics. The system uses a drug product having a targeted 8PEGA NC to obtain an MRI of the NC that is located in the targeted tissue, e.g., a tumor, and in this manner an image of the targeted tissues, as well as, the location, concentration, amount, and combinations and variations of these, of NC in the targeted tissue. The image provides data and information regarding shape, position and location, and combinations and variations of these, as well as other information, of the targeted tissue and NC. The image is then stored and transferred to a photoacoustic imaging device where the resolution of the MRI is enhanced. Based at least in part upon, or using, the enhanced PAI image of the NC and targeted tissues a custom laser delivery pattern for delivering the energy to activate the active agent on the NC is developed. The custom laser delivery pattern is then delivered to the targeted tissue. In this manner the very precise treatment of conditions can be performed. The combination of enhanced imaging, targeted NC, and predetermined laser delivery pattern provides the ability to very precisely remove tissue, including on the cellular level. This system and method essentially provide a cellular scalpel.

This system can be in an integrated unit. It can be in several different units in which the data from each is transferred to the others. In this manner the units can be configured in a network.

Additionally, the system can monitor the effects of the laser delivery patterns, and based upon historic data, for a particular condition, conditions or tissue types, refine and enhance the predetermined laser delivery pattern.

Additionally, the system provides the ability to conduct the various operations at different times. The 8PEG NC can remain in the targeted tissue, and remain active or viable as a theranostic material for about 12 hours to about 1 week, about 1 day to about 4 days, about 12 hours to about 3 days, and all values within these ranges, as well as, longer and shorter times.

### Example 35

An 8PEGA therapy NC having a dynamic therapy agent, e.g., active agent, that is activated upon exposure to sonic energy.

### Example 35A

An 8PEGA therapy NC having a sonodynamic therapy agent, e.g., active agent, that is activated upon exposure to sonic energy.

### Example 36

An 8PEGA theranostic NC having a dynamic therapy agent that is activated upon exposure to an energy source. The agent can be selected to be activated by sonic energy, light energy, or any electromagnetic energy source.

### Example 37

The systems and methods of Examples 32, 32A, 32B, 33 and 34, can further use models, and algorithms to further enhance the resolution of the images, the position, shape and location of the targeted tissues and NCs, and the laser delivery patterns.

### Example 38

The system of Examples 32, 32A, 32B, 33 and 34 are used to provide image increased layering and modeling of data. These layered and modeled images have value for diagnostics, therapeutics and theranostic purposes. This data, layered images and both can further be used with machine learning to provide enhanced systems of these Examples.

### Example 39

The system of Examples 32, 32A, 32B, 33 and 34 provide quantification of the 8PEGA NC in biological tissue (e.g. tumor area vs filtration organs).

### Example 40

An embodiment of an 8PEGA for application would entail the following: addition of a PS via DCC/NHS coupling reaction in DMF to yield about 1.5 PS per 8PEGA; conversion of amine arms to maleimides using NHS-PEG-MAL (2 kDa); addition of cysteine terminated peptides to anchor to 8PEGA through the well understood thiol-maleimide reaction; and where no further modification necessary for DWI using 8PEGA.

### Headings and Embodiments

It should be understood that the use of headings in this specification is for the purpose of clarity, and is not limiting in any way. Thus, the processes and disclosures described under a heading should be read in context with the entirely of this specification, including the various examples. The use of headings in this specification should not limit the scope of protection afford the present inventions.

The various embodiments of systems, therapies, processes, compositions, applications, and materials set forth in this specification may be used for various other fields and for various other activities, uses and embodiments. Additionally, these embodiments, for example, may be used with: existing systems, therapies, processes, compositions, applications, and materials; may be used with systems, therapies, processes, compositions, applications, and materials that may be developed in the future; and with systems, therapies, processes, compositions, applications, and materials that may be modified, in-part, based on the teachings of this specification. Further, the various embodiments and examples set forth in this specification may be used with each other, in whole or in part, and in different and various combinations. Thus, for example, the configurations provided in the various embodiments and examples of this specification may be used with each other. For example, the components of an embodiment having A, A′ and B and the components of an embodiment having A″, C and D can be used with each other in various combination, e.g., A, C, D, and A. A″ C and D, etc., in accordance with the teaching of this Specification. Thus, the scope of protection afforded the present inventions should not be limited to a particular embodiment, example, configuration or arrangement that is set forth in a particular embodiment, example, or in an embodiment in a particular figure.

The invention may be embodied in other forms than those specifically disclosed herein without departing from its spirit or essential characteristics. The described embodiments are to be considered in all respects only as illustrative and not restrictive.

1. A composition having therapy, imaging, diagnostic or theranostic applications, the composition comprising:
a plurality of nanoparticles, wherein the nanoparticles comprise a backbone material;
an active agent attached to the backbone; and,
wherein, the plurality of nanoparticles has a predetermined particle size distribution defined by a D10=n−5, D50=n, D90=x+5.

2. The composition of claim 1, wherein n is a number in the range of about 5 nm to about 25 nm.

3. The composition of claim 1, wherein n is a number in the range of 7 nm to 22 nm.

4. The composition of claim 1, wherein n is a number in the range of about 10 nm to about 20 nm.

5.-54. (canceled)

55. A method of providing a PDT, the method comprising:
obtaining data from an MRI of nanoparticles in a subject; and,
using the data, at least in part, to provide a PDT;
wherein the nanoparticles are essential free from heavy metals.

56. The method of claim 55, wherein the nanoparticles have less than 1 ppm heavy metals.

57. The method of claim 55, wherein the nanoparticles have less than 0.1 ppm heavy metals.

58. The method of claim 55, wherein the nanoparticles have less than 0.01 ppm heavy metals.

59. The method of claim 55, wherein the nanoparticles have less than 0.001 ppm heavy metals.

60. A method of providing a PDT, the method comprising:
obtaining data from an MRI of nanoparticles in a subject; and,
using the data, at least in part, to provide a PDT;
wherein the nanoparticles are essential free from gadolinium.

61.-64. (canceled)

65. A method of obtaining data for use in guiding therapeutic applications, the method comprising:
administering an imaging agent comprising a plurality of nanoparticles to a subject;
the nanoparticles being essentially from gadolinium; and,
performing a nuclear magnetic resonance scan of the subject after administration of the imaging agent;
wherein the nanoparticles are directly imaged; thereby providing an MRI of the nanoparticles and data related to the nanoparticles and the subject.

66. The method of claim 65, wherein the nanoparticles have less than 1 ppm gadolinium.

67. The method of claim 66, wherein the nanoparticles have less than 0.1 ppm gadolinium.

68.-86. (canceled)

87. A nuclear magnetic resonance imaging agent, the imaging agent comprising:
a plurality of nanoparticles that are essentially free from heavy metals;
the nanoparticles comprising PEG;
wherein the nanoparticles are capable of being directly imaged by a magnetic field generated by a magnetic resonance imaging system.

88.-90. (canceled)

91. An imaging agent comprising nanoparticles that are capable of being directly imaged by the magnetic field in a magnetic resonance imaging device, the nanoparticles comprising:
a nanoconstruct comprising a backbone material, wherein the backbone material is non-paramagnetic; and,
the nanoconstruct is capable of being directly imaged by a magnetic field.

92. The imaging agent of claim 91, wherein the nanoconstruct comprises about 2,000 to about 5,000 protons; and, wherein the nanoconstruct is less than 25 nm.

93. The imaging agent of claim 91, wherein the nanoconstruct comprises about 3,600 protons; and, wherein the nanoconstruct is less than 25 nm.

94. The imaging agent of claim 91, wherein the nanoconstruct comprises about 3,000 to about 5,000 protons; and, wherein the nanoconstruct is less than 20 nm.

95. The imaging agent of claim 91, wherein the nanoconstruct comprises about 5,000 to about 15,000 protons; and, wherein the nanoconstruct is less than 50 nm.

96.-102. (canceled)

103. An MRI system, the system configured to generate three magnetic fields; a first, a strong static magnetic field to create energy level differences in nuclei with spin angular momentum and gives rise to bulk nuclear magnetization; a second, a radio frequency field is used to tip the created nuclear magnetization so that it can be detected by RF coils; a third set of magnetic field gradients is used to spatially encode the signal to create a map of nuclear magnetization; the magnetic fields configured to generate an image of non-water protons present in an additive placed in a subject to be imaged; wherein the magnetic field gradients can be pulsed in a specific manner to sensitize the nuclei to motion due to flow or diffusion.

104. A method of imaging an 8PEGA imaging agent, the method comprising: providing a diffusion weighted spin-echo imaging sequence having a repetition time TR=500 ms, an echo time TE=200 ms, a pair of diffusion encoding gradients with amplitude G_{diff}=126 mT/m, duration δ=7.1 ms, and separation ∇=180 ms to generate a diffusion b value of 10¹⁰ s/m².

105. The method of claim 104, wherein the magnetic field gradients attenuate the MR signal intensity by S(b)=exp(−bD) where
$$b = {( γ δ {G}_{diff} )}^{2} ( Δ - \frac{δ}{3} ) .$$

106.-107. (canceled)

108. A method of obtaining an MRI image of a subject, the method comprising: administering an imaging agent to a subject; and obtaining an MRI image of the subject; wherein the MRI image comprising a direct image of non-water based protons contained in in the imaging agent.

109. (canceled)
